(19) United States
(12) Patent Application Publication
(10) Pub. No.: US 2003/0153042 A1
(43) Pub. Date: Aug. 14, 2003

(54) Expression of functional eukaryotic proteins

(75) Inventors: Frances H. Arnold, Pasadena, CA (US); Zhanglin Lin, Framingham, MA (US)

Correspondence Address: DARBY & DARBY P.C., Post Office Box 5257, New York, NY, 10150-5257, US

(73) Assignee: California Institute of Technology

(21) Appl. No.: 10/274,793

(22) Filed: Oct. 21, 2002

Related U.S. Application Data
(63) Continuation of application No. 09/654,493, filed on Sep. 1, 2000, now abandoned.
Application No. 09/654,493 is a continuation-in-part of application No. 09/538,591, filed on Mar. 27, 2000, now abandoned.
Application No. 09/538,591 is a continuation-in-part of application No. 09/247,232, filed on Feb. 9, 1999, now abandoned.
(60) Provisional application No. 60/106,840, filed on Nov. 3, 1998.
(60) Provisional application No. 60/094,403, filed on Jul. 28, 1998.

(51) Int. Cl. C12N 9/02; C07H 21/04; C12P 21/02; C12N 5/06

(52) U.S. Cl. 435/69.1; 435/189; 435/320.1; 435/325; 536/23.2

(57) ABSTRACT

This invention relates to the improved expression of evolved polynucleotide and polypeptide sequences encoding for eukaryotic enzymes, particularly peroxidase enzymes, in conventional or facile expression systems. Various methods for directed evolution of polynucleotide sequences can be used to obtain the improved sequences. The improved characteristics of the polypeptides or proteins generated in this maruer include improved folding, without formation of inclusion bodies, and retained functional activity. In a particular embodiment, the invention relates to improved expression of the horseradish peroxidase (HRP) gene and HRP enzymes. HRP mutants that are highly expressed, highly active, and/or thermostable, are disclosed.

This application is a continuation-in-part of U.S. application Nos. 09/538,591, filed Mar. 27, 2000, and 09/247,232 filed Feb. 9, 1999, which claims the priority of U.S. application Ser. No. 60/094,403 filed Jul. 28, 1998, and U.S. application Ser. No. 60/106,840 filed Nov. 3, 1998.

The Government has certain rights to this invention pursuant to Grant Nos. N0014-96-1-0340 and N00014-98-1-0657, awarded by the United States Navy.

### BACKGROUND OF THE INVENTION

1. Field of the Invention

The publications and reference materials noted herein and listed in the appended Bibliography are each incorporated by reference in their entirety.

This invention relates to methods for the selection and production of polynucleotides that encode functional polypeptides or proteins, especially eukaryotic proteins, and particularly in facile host cell expression systems. Facile expression systems include robust prokaryotic cells (e.g. bacteria) and eukaryotic systems (e.g. yeast). In particular, the invention concerns the recombinant production of expression-resistant functional eukaryotic proteins by host cells, in high yield, and without deactivation, denaturation, inclusion bodies, or other loss of structure or function. In preferred embodiments, the host cells secrete the expressed proteins. Preferred proteins of the invention include peroxidases and heme-containing proteins, such as horseradish peroxidase (HRP) and cytochrome c-peroxidase (CCP). Polynucleotides that encode and express these proteins in recombinant host cell expression systems are also encompassed by the invention.

2. Description of Related Art

The publications and reference materials noted herein and listed in the appended Bibliography are each incorporated by reference in their entirety. They are referenced numerically in the text and the Bibliography below.

Organisms having “eukaryotic” cells produce many proteins of interest. These are cells having a nucleus surrounded by its own membrane and containing DNA on structures called chromosomes. All multicellular organisms, such as humans and animals, and many single-cell animals, have eukaryotic cells. Other single-cell organisms, such as bacteria have “prokaryotic” cells. These cells have a primitive nucleus with DNA in a defined structure, but without chromosomes and a nuclear membrane that is characteristic of eukaryotes. Prokaryotic organisms are generally much easier and less costly to grow, maintain and manipulate than eukaryotic cells.

Genetic engineering and recombinant DNA and RNA technologies have made it possible to produce proteins, hormones and enzymes that are native to one organism, by using the cells of a different organism as “factories” or host cell expression systems. In particular, it is often desirable to express a protein of eukaryotic origin in a prokaryotic host cell, because the prokaryotes can be grown in large quantities of identical cells, to produce large amounts of the desired foreign protein. For example, certain human proteins may be useful as drugs if they can be supplied in sufficient quantity to patients who have a protein deficiency. Such proteins may not easily or ethically be obtained by isolating them from human cells, nor can they easily be made by direct chemical synthesis or by growing them in isolated tissue cultures. Other proteins and enzymes are useful in industry. For example, certain enzymes can break down food products, and are useful in laundry detergent. However, commercial applications require large amounts of protein and a high degree of quality control.

To solve some of these problems, recombinant genetic engineering techniques have been developed to use genetic machinery of other cells, such as bacteria and yeast, to produce human or other proteins. Selected genetic material, such as a polynucleotide that encodes a desired protein, is “recombined” with genetic material in a host cell, so that the host cell expresses the introduced foreign genetic material and produces the desired polypeptide or protein. Bacteria and yeast can be suitable host cells because they are easy and economical to grow and maintain in large quantities, and can be used to reliably and repeatably produce foreign proteins.

However, many proteins cannot easily be expressed in foreign host cells, including bacteria and yeast. Such expression-resistant polypeptides or proteins may not be expressed at all, or are expressed inefficiently, e.g. in low yield. The protein may be expressed, but can lose some or all of its or function. In some cases the expressed protein may lose some or all of its active folded structure, and may even become denatured or completely inactive. Expressed proteins may also be encapsulated inside inclusion bodies within a host cell. These are discrete particles or globules inside and separate from the rest of the cell, and which contain expressed protein, perhaps in agglomerated or inactive form. This makes it difficult to harvest the produced protein from the host cells, as the isolation and purification techniques can be difficult, inefficient, time-consuming and costly. Efforts to produce expression-resistant polypeptides in active or functional form and at relatively high yields have spanned many years and have been markedly unsuccessful. In particular, expression-resistant enzymes that are commercially important, such as peroxidase enzymes like horseradish peroxidase, have not been functionally expressed in reasonably high yield or in convenient, economical or facile host cells. These enzymes are instead produced in non-functional or inactive form, for example as inclusion bodies, and are laboriously manipulated and reconstituted to obtain active enzymes at relatively poor yields.

Some proteins that are made by cells can be secreted or delivered outside the cell, which can improve the yield and the efficiency of subsequent isolation and purification steps. However, many proteins are not naturally secreted, and are difficult to secrete artificially, for example because they contain chemical groups that do not easily cross the cell membrane. In particular, it is difficult to engineer a compatible protein and host cell system to secrete a protein that has a tendency to form inclusion bodies. Therefore, improved techniques for expressing foreign proteins are needed, particularly proteins of eukaryotic origin, and particularly recombinant proteins which can be secreted by host cells in high yield, and without loss of activity or function.

As discussed, a particular challenge when producing foreign proteins in a host cell expression system is the inability of many foreign proteins to fold properly into functional proteins when using common recombinant hosts such as E. coli and yeast (14-17). As a result, the polypeptide chains that are produced in a recombinant host cell system are often degraded upon synthesis or accumulate in inclusion bodies. This is particularly true for eukaryotic proteins that contain disulfide bonds or are glycosylated in the native form. The underlying reasons, which are not clearly understood and are probably multifactorial, may include the “unnatural” recombinant environments in which the proteins accumulate (48) and the lack of proper folding cofactors such as molecular chaperones in the E. coil host (16). Additionally, glycosylation has been implicated in protein folding in eukaryotic organisms (49), which function is absent in bacteria.

The folding problem presents a challenging roadblock to the large-scale production of proteins for pharmaceutical or industrial applications. The lack of high-efficiency functional expression systems has also become one of the bottlenecks in applying directed evolution techniques for optimizing proteins and reaction conditions for desired uses. Employing random mutagenesis and gene recombination followed by screening or selection, directed evolution has been successfully applied to improve a variety of enzyme properties, such as substrate specificity, activity in organic solvents, and stability at high temperatures, which are often critical for industrial applications (18). Eukaryotic enzymes have a myriad of existing and potential applications, but improvement of these proteins by directed evolution had been limited by the inability to functionally express them in a facile recombinant host.

For example, the difficulty of expressing peroxidase enzymes in a facile expression host has posed at least two technical challenges for realizing the potential of peroxidases as biocatalysts. First, efforts to modify these enzymes for industrial applications by protein engineering methods have been impeded. Directed evolution, for example, exploits expression in a host such as E. coli or S. cerevisiae, organisms in which large libraries of mutants or variants can be made. Second, the lack of efficient expression in an appropriate foreign (heterologous) host prevents the mass production of some of these proteins on an economical scale.

One way to obtain the active form of recombinantly expressed proteins is by refolding them in vitro from inclusion bodies, but these processes are often laborious and inefficient (14-16). Additionally, this is not a viable option for directed evolution in which screening of tens of thousands of mutants is required. A more advantageous means to resolve the problem may be to identify mutations in a target gene that can facilitate folding in host environments. Evidence from a number of studies increasingly suggests that certain residues of an amino acid sequence have a profound influence on the folding per se of the protein. Thus, it would be highly advantageous if scientists could identify mutations in a target gene that facilitate folding in the host environrnent. This may avoid the inclusion body obstacle, but such techniques require the discovery, identification, and use of particular beneficial mutants.

For example, a series of studies by King and coworkers have shown that several single amino acid substitutions interfered with the productive folding of the phage p22 tailspike protein at restrictive temperature in vivo, and that second-site suppresser mutations were able to rescue the defective folding mutants (19). In another study, the replacement of tyrosine 35 with leucine in bovine pancreatic trypsin inhibitor (BPTI) eliminated kinetic traps in the folding pathway in vitro (20). Furthermore, it was reported that several mutants of human interleukin 1β, created by cassette mutagenesis of a few selected residues, were expressed in E. coli in soluble form, while the wild type was largely insoluble and formed inclusion bodies (21). In a separate study, a single site-directed mutation was found to improve the folding yield of a recombinant antibody (22).

It is difficult to predict which residues are critical for protein function or stability, let alone folding. Thus, it would be advantageous if there was a method for systematically searching for beneficial mutations that affect the folding and expression of proteins, without compromising biological activity. Directed evolution techniques may prove useful in the accomplishment of this goal. This evolutionary approach uses simultaneous random mutagenesis and recombination, to generate a variant having an improved desirable property over the existing wild type protein. Point mutations are generated due to the intrinsic infidelity of Taq-based polymerase chain reactions (PCR) associated with reassembly of nucleic acid sequences. In one example, Stemmer and coworkers applied this technique to the gene encoding for green fluorescence protein (GFP), which resulted in a protein that folded better than the wild type in E. coli (23).

One group of proteins of particular interest are heme proteins, that is, they have iron-containing heme groups. These proteins have many biological and biochemical uses, and include certain enzymes called peroxidases, which are enzymes that facilitate oxidation or reduction reactions in which a peroxide (e.g. hydrogen peroxide) is one of the reactants. Peroxides are compounds, other than molecular O₂, in which oxygen atoms are joined to each other. For example, the heme enzyme horseradish peroxidase (HRP) is widely used as a reporter in diagnostic assays. HRP catalyzes a reaction in which starting materials or substrates are chemically combined in the presence of a peroxide, such as hydrogen peroxide (H₂O₂), with water (H₂O) as a byproduct. This reaction can be exploited to indicate whether another reaction of interest has occurred, or whether certain materials, such as HRP starting materials, are present in a mixture or sample. It would be beneficial to provide a means of producing large quantities of HRP, and other heme or peroxidase enzymes, using efficient and cost-effective systems such as prokaryotic expression systems. However, native HRP contains four disulfides and is highly glycosylated (˜21%), although the carbohydrate moiety has no apparent effect on the activity or stability (24). As a consequence, previous attempts to express HRP in bacteria have yielded inclusion bodies, with no functional expression (25-27). Successful expression in yeast has also not been achieved prior to this invention.

Accordingly, there is a need to develop new and improved methods for expressing proteins which ordinarily have difficulty being expressed in order to obviate the need for laborious in vitro folding protocols. In particular, there is a need for protein expression methods which are well-suited for use in connection with directed evolution techniques.

In particular, this invention describes methods for screening libraries of HRP mutants produced by error-prone PCR and DNA shuffling to identify mutations that facilitate functional expression in bacteria (E. coli, B. subtilis) and yeast (S. cerevisiae). In one exemplary embodiment, the variant of the invention is a functional and active horseradish peroxidase (HRP) that is expressed in E. coli without inclusion bodies at levels of about 110 μg/L. This is comparable to amounts previously obtained from much more costly, time-consuming and laborious in vitro refolding techniques used to recover other HRP enzymes from inclusion bodies.

### SUMMARY OF THE INVENTION

The observed constraints on the use of native proteins are thought to be a consequence of evolution. Proteins have evolved in the context and environment of a living organism, to carry out specific biological functions under conditions conducive to life—not in the laboratory or under industrial conditions. In some cases, evolution may favor or even require less than optimally efficient enzymes. The output, efficiency, working conditions, stability and other properties of known expression systems are not thought to be unalterable, nor are they limitations which should be seen as intrinsic to the nature of cellular expression systems. It is possible that the proteins used in these systems can be evolved in vitro, or that analogous proteins can be otherwise developed, to alter or enhance the protein's properties, for example, to obtain much more efficient expression, folding, and secretion, while maintaining activity of the protein. Improved proteins can also be obtained by screening cultures of native organisms or expressed gene libraries (17).

Many proteins, when expressed using facile expression systems (e.g., E. coli) result in inclusion bodies or are inactive due to an inability to properly fold. The invention takes advantage of directed evolution techniques to create novel polynucleotides encoding for mutated functional proteins which have an increased ability to be produced in an expression system, without inactivation or inclusion bodies. In preferred embodiments the protein is secreted outside of the cell.

There are several advantages to secreting proteins from bacteria or yeast into the culture media, since in many cases desired substrates cannot readily pass through the membranes of E. coli. Secretion can facilitate screening in directed evolution studies, because, by allowing the secreted enzyme to catalyze a reaction in the culture medium, substrates that cannot enter the cells can be used. It can also significantly simplify the production of recombinant proteins, as the culture supernatant is largely free of contaminating substances, if the secretion level is high enough. Nonetheless, secretion of proteins into culture media remains a difficult task, particularly for enzymes that contain bulky prosthetic groups such as heme.

This problem can be solved by using a suitable signal peptide, such as the signal from the pectate lyase B (PelB) of Erwinia carotovora (40), to efficiently direct the secretion of a peroxidase such as HRP or CCP into the culture medium. This signal peptide is also generally applicable to other proteins containing heme prosthetic groups, such as cytochrome P450 enzymes and other peroxidases.

According to one embodiment of the invention, directed evolution or random mutagenesis is used to produce in vitro proteins which readily fold after expression, in yeast, e.g., S. cerevisiae. Prokaryotic expression systems such as E. coli may also be used. These proteins are easily secreted outside the host cell in quantities expected for proteins produced by such expression systems. Furthermore, activity of these proteins is not compromised by the mutagenic step after appropriate selection is made.

Thus, the invention provides a method for improving the expression of a polynucleotide encoding peroxidase enzymes by using directed evolution, and polynucleotides encoding for variant horseradish peroxidase which have improved expression in conventional expression systems.

The above features and many other attendant advantages of the invention will become better understood by reference to the following detailed description when taken in conjunction with the accompanying drawings.

### BRIEF DESCRIPTION OF THE DRAWINGS

FIG. 1 is a schematic map of an E. coli HRP expression vector pETHRP, the plasmid pETpelBHRP. The HRP gene (with an extra methionine residue at the N-terminus) was inserted into pET-22b(+), immediately downstream of the signal sequence from the pectate lyase B (PelB) of Erwinia carotovora for periplasmic localization. Expression is under the control of the T7 promoter.

FIG. 2 shows the nucleic acid and amino acid sequences of the pelB signal peptide [SEQ ID NO: 1 and SEQ ID NO: 2].

FIG. 3 shows a nucleotide and amino acid sequence encoding a recombinant wild-type HRP enzyme designated HRP1A6 ([SEQ. ID NO. 3 and SEQ. ID. NO. 4]).

FIG. 4 is a map of the expression vector pETpelBHRP1A6.

FIG. 5A shows the relative activities of wild-type HRP and recombinant wild-type HRP1A6. FIG. 5B shows a representative landscape of first generation HRP mutants sorted by activity in descending order. Activities are normalized to that of wild-type.

FIG. 6 shows activity levels of HRP1A6 at various ITPG concentrations.

FIG. 7 is a representation of the structure of HRP. This figure was generated from published HRP coordinates (47), using Insight II software (Molecular Biosystems).

FIG. 8 is a map of the expression vector pYEXS1-HRP containing a coding sequence for HRP cloned into the secretion plasmid pYEX-S1.

FIG. 9 shows the activity levels of HRP1A6 and three mutants obtained by directed evolution in S. cerevisiae: HRP1-77E2, HRP1-117G4, and HRP2-28D6. In this example, HRP1A6 was the parent of HRP1-77E2 and HRP1-117G4, while HRP1-117G4 was the parent of HRP2-28D6.

FIG. 10 shows residual activity of several HRP mutants as a function of temperature, in a thermal inactivation curve that indicates the relative thermostability of the mutants.

FIG. 11 shows the residual activity of several HRP mutants as a function of hydrogen peroxide concentration, in a titration curve that indicates the relative ability of the mutants to resist degradation in the presence of hydrogen peroxide.

FIG. 12 shows a nucleotide and amino acid sequence encoding an HRP enzyme variant designated HRP1-77E2 ([SEQ ID NO:5 and SEQ ID NO:6]).

FIG. 13 shows a nucleotide and amino acid sequence encoding an HRP enzyme variant designated HRP1-4B6 ([SEQ ID NO:7 and SEQ ID NO:8]).

FIG. 14 shows a nucleotide and amino acid sequence encoding an HRP enzyme variant designated HRP1-28B11 ([SEQ ID NO:9 and SEQ ID NO:10]).

FIG. 15 shows a nucleotide and amino acid sequence encoding an HRP enzyme variant designated HRP1-24D11 ([SEQ ID NO:11 and SEQ ID NO:12]).

FIG. 16 shows a nucleotide and amino acid sequence encoding an HRP enzyme variant designated HRP1-117G4 ([SEQ ID NO:13 and SEQ ID NO:14]).

FIG. 17 is a map of the yeast cytochrome c peroxidase expression vector pETCCP

FIG. 18 shows a nucleotide and amino acid sequence encoding an HRP enzyme variant designated HRP1-80C12 ([SEQ ID NO:17 and SEQ ID NO:18]).

FIG. 19 shows a nucleotide and amino acid sequence encoding an HRP enzyme variant designated HRP2-28D6 ([SEQ ID NO:19 and SEQ ID NO:20]).

FIG. 20 shows a nucleotide and amino acid sequence encoding an HRP enzyme variant designated HRP2-13A10 ([SEQ ID NO:21 and SEQ ID NO:22]).

FIG. 21 shows a nucleotide and amino acid sequence encoding an HRP enzyme variant designated HRP3-17E12 ([SEQ ID NO:23 and SEQ ID NO:24]).

FIG. 22 shows the total and residual HRP activity of HRP1A6 (recombinant wild-type), and mutants HRP1-117G4, HRP1-77E12, HRP1-80C12, HRP2-13A10, HRP2-28D6, and HRP3-17E12.

FIG. 23 shows amino acid substitutions in mutants HRP2-13A10 and HRP3-17E12.

FIG. 24 is a schematic map of the yeast P. pastoris expression vector pPICZ_B-HRP.

FIG. 25 shows HRP activity profiles for representative S. cerevisiae cultures. Activities are normalized with respect to OD₆₀₀.

FIG. 26 shows HRP activity profiles for representative P. pastoris cultures. Activities are normalized with respect to OD₆₀₀.

FIG. 27 shows electrophoresis of purified HRP-C variants.

FIG. 28 shows the lineage of HRP mutations obtained by directed evolution. Nucleotide substitutions are shown in parentheses, following corresponding amino acid substitutions. Synonymous mutations are in italics. New mutations in each generation are indicated by an asterisk (*).

### DETAILED DESCRIPTION OF THE INVENTION

This invention concerns methods for improving the expression of proteins using conventional expression systems, which proteins would ordinarily result in inclusion bodies or are degraded upon synthesis due to an inability to fold properly in the environment of the expression system.

Definitions

As used herein, “about” or “approximately” shall mean within 50 percent, preferably within 20 percent, more preferably within 5 percent, and even more preferably within 5 percent of a given value or range.

The term “polymer” means any substance or compound that is composed of two or more building blocks (‘mers’) that are repetitively linked to each other. For example, a “dimer” is a compound in which two building blocks have been joined together.

A “protein” or “polypeptide”, which terms are used interchangeably herein, comprises one or more chains of chemical building blocks called amino acids that are linked together by chemical bonds called peptide bonds.

An “enzyme” means any substance, preferably composed wholly or largely of protein, that catalyzes or promotes, more or less specifically, one or more chemical or biochemical reactions. The term “enzyme” can also refer to a catalytic polynucleotide (e.g. RNA or DNA). A “test” enzyme is a substance that is tested to determine whether it has properties of an enzyme.

Proteins and enzymes can be made in a host cell using instructions in DNA and RNA, according to the genetic code. “Transcription” is the process by which a DNA sequence or gene having instructions for a particular protein or enzyme is “transcribed” into a corresponding sequence of RNA. “Translation” is the process by which the RNA sequence is “translated” into the sequence of amino acids which form the protein or enzyme.

A “native” or “wild-type” protein, enzyme, polynucleotide, gene, or cell, means a protein, enzyme, polynucleotide, gene, or cell that occurs in nature.

A “parent” protein, enzyme, polynucleotide, gene, or cell, is any protein, enzyme, polynucleotide, gene, or cell, from which any other protein, enzyme, polynucleotide, gene, or cell, is derived or made, using any methods, tools or techniques, and whether or not the parent is itself native or mutant. A parent polynucleotide or gene can encode for a parent protein or enzyme.

A “mutant”, “variant” or “modified” protein, enzyme, polynucleotide, gene, or cell, means a protein, enzyme, polynucleotide, gene, or cell, that has been altered or derived, or is in some way different or changed, from a parent protein, enzyme, polynucleotide, gene, or cell. A mutant or modified protein or enzyme is usually, although not necessarily, expressed from a mutant polynucleotide or gene.

A “mutation” means any process or mechanism resulting in a mutant protein, enzyme, polynucleotide, gene, or cell. This includes any mutation in which a protein, enzyme, polynucleotide, or gene sequence is altered, any protein, enzyme, polynucleotide, or gene sequence arising from a mutation, any expression product (e.g. protein or enzyme) expressed from a mutated polynucleotide gene sequence, and any detectable change in a cell arising from such a mutation.

Regarding genetic material, “mutant” and “mutation” includes polynucleotide alterations arising within a protein-encoding region of a gene as well as alterations in regions outside of a protein-encoding sequence, such as, but not limited to, regulatory sequences. “Mutant” also includes a “silent” mutant and “sequence-conservative variants”, which is a mutant polynucleotide sequence that, upon translation, is not reflected in an altered amino acid sequence. Such silent mutations can occur when one amino acid corresponds to more than one codon.

“Function-conservative variants” are proteins or enzymes in which a given amino acid residue has been changed without altering overall conformation and function of the protein or enzyme, including, but not limited to, replacement of an amino acid with one having similar properties (such as, for example, acidic, basic, hydrophobic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST or FASTA algorithms, preferably at least 75%, most preferably at least 85%, and even more preferably at least 90%, and which has the same or substantially similar properties or functions as the native or parent protein or enzyme to which it is compared.

A “functional” protein or enzyme is capable of displaying biological activity, such as, for example, participating in a designated biochemical reaction. Generally, a screening test can be used to detect and/or evaluate whether a protein is functional or not.

A “property” of a protein or enzyme, wild-type or mutated, means a feature, preferably detectable in a screening test, associated with the protein. Protein properties include, but are not limited to, the ability of the protein to fold correctly, the stability of the protein in a certain media and/or over time, the expression level or yield of a protein expressed by a host cell, functionality (i.e., whether the protein is functional or non-functional), and, in the case of a enzyme, enzyme activity.

The “activity” of an enzyme is a measure of its ability to catalyze a reaction, and may be expressed as the rate at which the product of the reaction is produced. For example, enzyme activity can be represented as the amount of product produced per unit of time, per unit (e.g. concentration or weight) of enzyme.

The “stability” of an enzyme means its ability to function, over time, in a particular environment or under particular conditions. One way to evaluate stability is to assess its ability to resist a loss of activity over time, under given conditions. Enzyme stability can also be evaluated in other ways, for example, by determining the relative degree to which the enzyme is in a folded or unfolded state. Thus, one enzyme is more stable than another, or has improved stability, when it is more resistant than the other enzyme to a loss of activity under the same conditions, is more resistant to unfolding, or is more durable by any suitable measure. For example, a more “thermally stable” or “thermostable” enzyme is one that is more resistant to loss of structure (unfolding) or function (enzyme activity) when exposed to heat or an elevated temperature. One way to evaluate this is to determine the “melting temperature” or Tₘ for the protein. The melting temperature, also called a midpoint, is the temperature at which half of the protein is unfolded from its fully folded state. This midpoint is typically determined by calculating the midpoint of a titration curve that plots protein unfolding as a function of temperature. Thus, a protein with a higher Tₘ requires more heat to cause unfolding and is more stable or more thermostable. Stated another way, a protein with a higher Tₘ indicates that fewer molecules of that protein are unfolded at the same temperature as a protein with a lower Tₘ, again meaning that the protein which is more resistant to unfolding is more stable (it has less unfolding at the same temperature). Another measure of stability is T_{½}, which is the transition midpoint of the inactivation curve of the protein as a function of temperature. T_{½} is the temperature at which the protein loses half of its activity. Thus, a protein with a higher T_{½} requires more heat to deactivate it, and is more stable or more thermostable. Stated another way, a protein with a higher T_{½} indicates that fewer molecules of that protein are inactive at the same temperature as a protein with a lower T_{½}, again meaning that the protein which is more resistant to deactivation is more stable (it has more activity at the same temperature). These assays are also called “thermal shift” assays, because the inactivation or unfolding curve, plotted against temperature, is “shifted” to higher or lower temperatures when stability increases or decreases. Thermostability can also be measured in other ways. For example, a longer half-life (t_{½}) for the enzyme's activity at elevated temperature is an indication of thermostability.

The term “substrate” means any substance or compound that is converted or meant to be converted into another compound by the action of an enzyme catalyst. The term includes aromatic and aliphatic compounds, and includes not only a single compound, but also combinations of compounds, such as solutions, mixtures and other materials which contain at least one substrate.

The term “cofactor” means any non-protein substance that is necessary or beneficial to the activity of an enzyme. A “coenzyme” means a cofactor that interacts directly with and serves to promote a reaction catalyzed by an enzyme. Many coenzymes serve as carriers. For example, NAD⁺ and NADP⁺ carry hydrogen atoms from one enzyme to another. An “ancillary protein” means any protein substance that is necessary or beneficial to the activity of an enzyme.

An “oxidation reaction” or “oxygenation reaction”, as used herein, is a chemical or biochemical reaction involving the addition of oxygen to a substrate, to form an oxygenated or oxidized substrate or product. An oxidation reaction is typically accompanied by a reduction reaction (hence the term “redox” reaction, for oxidation and reduction). A compound is “oxidized” when it receives oxygen or loses electrons. A compound is “reduced” when it loses oxygen or gains electrons. An oxidation reaction can also be called an “electron transfer reaction” and encompass the loss or gain of electrons (e.g. oxygen) or protons (e.g. hydrogen) from a substance.

The terms “oxygen donor”, “oxidizing agent” and “oxidant” mean a substance, molecule or compound which donates oxygen to a substrate in an oxidation reaction. Typically, the oxygen donor is reduced (accepts electrons). Exemplary oxygen donors, which are not limiting, include molecular oxygen or dioxygen (O₂) and peroxides, including alkyl peroxides such as t-butyl peroxide, and most preferably hydrogen peroxide (H₂O₂). A peroxide is any compound having two oxygen atoms bound to each other.

An “oxidation enzyme” is an enzyme that catalyzes one or more oxidation reactions, typically by adding, inserting, contributing or transferring oxygen from a source or donor to a substrate. Such enzymes are also called oxidoreductases or redox enzymes, and encompasses oxygenases, hydrogenases or reductases, oxidases and peroxidases. An “oxidase” is an oxidation enzyme that catalyzes a reaction in which molecular oxygen (dioxygen or O₂) is reduced, for example by donating electrons to (or receiving protons from) hydrogen.

A “luminescent” substance means any substance which produces detectable electromagnetic radiation, or a change in electromagnetic radiation, most notably visible light, by any mechanism, including color change, UV absorbance, fluorescence and phosphorescence. Preferably, a luminescent substance according to the invention produces a detectable color, fluorescence or UV absorbance.

The term “chemiluminescent agent” means any substance which enhances the detectability of a luminescent (e.g., fluorescent) signal, for example by increasing the strength or lifetime of the signal. One exemplary and preferred chemiluminescent agent is 5-amino-2,3-dihydro-1,4-phthalazinedione (luminol) and analogs. Other chemiluminescent agents include 1,2-dioxetanes such as tetramethyl-1,2-dioxetane (TMD), 1,2-dioxetanones, and 1,2-dioxetanediones.

“DNA” (deoxyribonucleic acid) means any chain or sequence of the chemical building blocks adenine (A), guanine (G), cytosine (C) and thymine (T), called nucleotide bases, that are linked together on a deoxyribose sugar backbone. DNA can have one strand of nucleotide bases, or two complimentary strands which may form a double helix structure. “RNA” (ribonucleic acid) means any chain or sequence of the chemical building blocks adenine (A), guanine (G), cytosine (C) and uracil (U), called nucleotide bases, that are linked together on a ribose sugar backbone. RNA typically has one strand of nucleotide bases.

A “polynucleotide” or “nucleotide sequence” is a series of nucleotide bases (also called “nucleotides”) in DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide (although only sense stands are being represented herein). This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The polynucleotides herein may be flanked by natural regulatory sequences, or may be associated with heterologous sequences, including promoters, enhancers, response elements, signal sequences, polyadenylation sequences, introns, 5′- and 3′- non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A “codon” is a triplet of nucleotides corresponding to an amino acid. Each amino acid is represented in DNA or RNA by one or more codons. The genetic code has some redundancy, also called degeneracy, meaning that most amino acids have more than one corresponding codon. For example, the amino acid lysine (Lys) can be coded by the nucleotide triplet or codon AAA or by the codon AAG.

The “reading frame” describes the way that a nucleotide sequence is grouped into codons. Because the nucleotides in DNA and RNA sequences are read in groups of three for protein production, it is important to begin reading the sequence at the correct amino acid, so that the correct triplets are read.

A “coding sequence” or a sequence “encoding” a polypeptide, protein or enzyme is a nucleotide sequence that, when expressed, results in the production of that polypeptide, protein or enzyme, i.e., the nucleotide sequence encodes an amino acid sequence for that polypeptide, protein or enzyme. A coding sequence is “under the control” of transcriptional and translational control sequences in a cell when RNA polymerase transcribes the coding sequence into mRNA, which is then trans-RNA spliced and translated into the protein encoded by the coding sequence. Preferably, the coding sequence is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. A gene encoding a protein of the invention for use in an expression system, whether genomic DNA or cDNA, can be isolated from any source, particularly from a human cDNA or genomic library. Methods for obtaining genes are well known in the art, e.g., Sambrook et al. (32).

Any animal cell potentially can serve as the nucleic acid source for the molecular cloning of the gene of interest. The DNA may be obtained by standard procedures known in the art, such as from cloned DNA (e.g., a DNA “library”), from cDNA library prepared from tissues with high level expression of the protein, by chemical synthesis, by cDNA cloning, or by the cloning of genomic DNA, or fragments thereof, purified from the desired cell (32, 51). Clones derived from genomic DNA may contain regulatory and intron DNA regions in addition to coding regions; clones derived from cDNA will not contain intron sequences.

In the molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

A transcriptional or translational “control sequence” is a DNA regulatory sequence, such as a promoter, enhancer, terminator, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining this invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. As described above, promoter DNA is a DNA sequence which initiates, regulates, or otherwise mediates or controls the expression of the coding DNA. A promoter may be “inducible”, meaning that it is influenced by the presence or amount of another compound (an “inducer”). For example, an inducible promoter includes those which initiate or increase the expression of a downstream coding sequence in the presence of a particular inducer compound. A “leaky” inducible promoter is a promoter that provides a high expression level in the presence of an inducer compound and a comparatively very low expression level, and at minimum a detectable expression level, in the absence of the inducer.

A “signal sequence” is included at the beginning of the coding sequence of a protein to be expressed in the periplasmic space, or outside the cell. This sequence encodes a signal peptide, N-terminal to the mature polypeptide, that directs the host cell to translocate the polypeptide. The term “translocation signal sequence” is also used to refer to a signal sequence. Translocation signal sequences can be found associated with a variety of proteins native to eukaryotes and prokaryotes, and are often functional in both types of organisms. Proteins of the invention may be further modified and improved by adding a sequence which directs the secretion of the protein outside the host cell. The addition of the signal sequence does not interfere with the folding of the secreted protein, and evidence thereof is easily tested for using techniques known in the art and depending on the protein (e.g., tests for activity of a given protein after modification).

Preferred signal sequences of the invention include the pelB signal sequence, which normally directs a protein to the periplasmic space between the inner and outer membranes of bacteria. Other signal sequences include, for example ompA and ompT (65). For yeast, a suitable signal sequence includes the α-subunit of K. lactis toxin. The signal sequence is ligated upstream of the nucleotide sequence encoding the protein, such that the sequence is present at the N-terminus of the protein after expression. Conventional cloning techniques can be used as described. Some routine experimentation within the scope of one skilled in the art may be necessary to optimize addition of the signal sequence to any given protein.

Polynucleotides are “hybridizable” to each other when at least one strand of one polynucleotide can anneal to another polynucleotide under defined stringency conditions. Stringency of hybridization is determined, e.g., by a) the temperature at which hybridization and/or washing is performed, and b) the ionic strength and polarity (e.g., formamide) of the hybridization and washing solutions, as well as other parameters. Hybridization requires that the two polynucleotides contain substantially complementary sequences; depending on the stringency of hybridization, however, mismatches may be tolerated. Typically, hybridization of two sequences at high stringency (such as, for example, in an aqueous solution of 0.5× SSC at 65° C.) requires that the sequences exhibit some high degree of complementarity over their entire sequence. Conditions of intermediate stringency (such as, for example, an aqueous solution of 2× SSC at 65° C.) and low stringency (such as, for example, an aqueous solution of 2× SSC at 55° C.), require correspondingly less overall complementarity between the hybridizing sequences. (1× SSC is 0.15 M NaCl, 0.015 M Na citrate.) Polynucleotides that “hybridize” to the polynucleotides herein may be of any length. In one embodiment, such polynucleotides are at least 10, preferably at least 15 and most preferably at least 20 nucleotides long. In another embodiment, polynucleotides that hybridizes are of about the same length. In another embodiment, polynucleotides that hybridize include those which anneal under suitable stringency conditions and which encode polypeptides or enzymes having the same function, such as the ability to catalyze an oxidation, oxygenase, or coupling reaction of the invention.

The term “DNA reassembly” is used when recombination occurs between identical sequences. “DNA shuffling” refers herein to a group of in vitro and in vivo methods involving recombination of nucleic acid species. Such methods can be employed to generate polynucleotide molecules having variant sequences of the invention.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme.

The term “expression system” means a host cell and compatible vector under suitable conditions, e.g. for the expression of a protein coded for by foreign DNA carried by the vector and introduced to the host cell. Common expression systems include bacteria (e.g. E. coli and B. subtilis) or yeast (e.g. S. cerevisiae) host cells and plasmid vectors, and insect host cells and Baculovirus vectors. As used herein, a “facile expression system” means any expression system that is foreign or heterologous to a selected polynucleotide or polypeptide, and which employs host cells that can be grown or maintained more advantageously than cells that are native or heterologous to the selected polynucleotide or polypeptide, or which can produce the polypeptide more efficiently or in higher yield. For example, the use of robust prokaryotic cells to express a protein of eukaryotic origin would be a facile expression system. Preferred facile expression systems include E. coli, B. subtilis and S. cerevisiae host cells and any suitable vector.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.”

A common type of vector is a “plasmid”, which generally is a self-contained molecule of double-stranded DNA, that can readily accept additional (foreign) DNA and which can readily introduced into a suitable host cell. A plasmid vector often contains coding DNA and promoter DNA and has one or more restriction sites suitable for inserting foreign DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes. Preferred vectors are described in the Examples, and include without limitations pcWori, pET-26b(+), pXTD14, pYEX-S1, pMAL, and pET22-b(+). Other vectors may be employed as desired by one skilled in the art. Routine experimentation in biotechnology can be used to determine which vectors are best suited for used with the invention, if different than as described in the Examples. In general, the choice of vector depends on the size of the polynucleotide sequence and the host cell to be employed in the methods of this invention.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. A polynucleotide or polypeptide is expressed recombinantly, for example, when it is expressed or produced in a foreign host cell under the control of a foreign or native promoter, or in a native host cell under the control of a foreign promoter.

A polynucleotide or polypeptide is “over-expressed” when it is expressed or produced in an amount or yield that is substantially higher than a given base-line yield, e.g. a yield that occurs in nature. For example, a polypeptide is over-expressed when the yield is substantially greater than the normal, average or base-line yield of the native polypolypeptide in native host cells under given conditions, for example conditions suitable to the life cycle of the native host cells. Over-expression of a polypeptide can be achieved, for example, by altering any one or more of: (a) the growth or living conditions of the host cells; (b) the polynucleotide encoding the polypeptide to be over-expressed; (c) the promoter used to control expression of the polynucleotide; and (d) the host cells themselves. This is a relative, and thus “over-expression” can also be used to compare or distinguish the expression level of one polypeptide to another, without regard for whether either polypeptide is a native polypeptide or is encoded by a native polynucleotide. Typically, over-expression means a yield that is at least about two times a normal, average or given base-line yield. Thus, a polypeptide is over-expressed when it is produced in an amount or yield that is substantially higher than the amount or yield of a parent polypeptide or under parent conditions. Likewise, a polypeptide is “under-expressed” when it is produced in an amount or yield that is substantially lower than the amount or yield of a parent polypeptide or under parent conditions, e.g. at least half the base-line yield. In this context, the expression level or yield refers to the amount or concentration of polynucleotide that is expressed, or polypeptide that is produced (i.e. expression product), whether or not in an active or functional form. As one example, a polynucleotide or polypeptide may be said to be under-expressed when it is expressed in detectable amounts under the control of an inducible promoter, but without induction, i.e. in the absence of an inducer compound.

An expression product can be characterized as intracellular, extracellular or secreted. The term “intracellular” means something that is inside a cell. The term “extracellular” means something that is outside a cell. A substance is “secreted” by a cell if it delivered to the periplasm or outside the cell, from somewhere on or inside the cell.

As used herein, the terms “expression-resistant polypeptide” and “resistant to functional expression” are synonymous and refer to a polypeptide that is difficult to functionally express in selected host cells. For example, an expression-resistant polypeptide is not produced, or is produced in very low yield or in non-functional form, when a polynucleotide encoding that polypeptide is transformed or introduced into host cells, e.g. into a facile host cell expression system.

These polypeptides include, for example, those which have disulfide bridges, which are composed of mutiple subunits, or which require glycosylation. Expression-resistant polypeptides also include those which are sensitive to folding and unfolding conditions, particularly intracellular conditions (inside the cell), such as temperature, pH, protein concentration, and the presence or absence of certain cofactors, coenzymes, ancillary proteins, etc. Expression-resistant polypeptides also include polypeptides that are encoded by polynucleotides which are sensitive to particular promoters or signal sequences in particular expression systems. In addition, expression-resistant polypeptides include those which tend to agglomerate, form inclusion bodies, or which are produced in a non-active or unfolded form. Particularly suitable for use as expression-resistant parent polypeptides in the invention are polypeptides that are inactive (e.g. they agglomerate, etc.) when produced at a high yield (e.g. when they are over-expressed), but which are active (e.g. they do not agglomerate, etc.) when produced at a very low yield (e.g. when they are under-expressed). These include, for example, polypeptides that: (a) tend to agglomerate, form inclusion bodies, or are inactive or unfolded, when expressed in the presence of an inducer, by a polynucleotide that is under the control of an inducible promoter; and (b) tend not to agglomerate, etc., and are active, when expressed without inducer, by a polynucleotide that is under the control of the inducible promoter. Such promoters are known and can be called “leaky” promoters. Polypeptides that include, incorporate or are associated with heme groups are also examples of expression-resistant polypeptides. Particular expression-resistant polypeptides of the invention are peroxidase enzymes, such as horseradish peroxidase enzymes. An “expression-resistant polynucleotide” is a polynucleotide that encodes an expression-resistant polypeptide.

“Isolation” or “purification” of a polypeptide or enzyme refers to the derivation of the polypeptide by removing it from its original environment (for example, from its natural environment if it is naturally occurring, or form the host cell if it is produced by recombinant DNA methods). Methods for polypeptide purification are well-known in the art, including, without limitation, preparative disc-gel electrophoresis, isoelectric focusing, HPLC, reversed-phase HPLC, gel filtration, ion exchange and partition chromatography, and countercurrent distribution. For some purposes, it is preferable to produce the polypeptide in a recombinant system in which the protein contains an additional sequence tag that facilitates purification, such as, but not limited to, a polyhistidine sequence. The polypeptide can then be purified from a crude lysate of the host cell by chromatography on an appropriate solid-phase matrix. Alternatively, antibodies produced against the protein or against peptides derived therefrom can be used as purification reagents. Other purification methods are possible. A purified polynucleotide or polypeptide may contain less than about 50%, preferably less than about 75%, and most preferably less than about 90%, of the cellular components with which it was originally associated. A “substantially pure” enzyme indicates the highest degree of purity which can be achieved using conventional purification techniques known in the art.

The general genetic engineering tools and techniques discussed here, including transformation and expression, the use of host cells, vectors, expression systems, etc., are well known in the art.

Mutagenesis and Directed Evolution of Proteins.

To improve the expression of proteins using conventional expression systems, the invention makes the unexpected discovery that directed evolution can be used to generate mutant libraries of polynucleotides which, when expressed using conventional or facile expression systems, result in functional proteins having normal or even higher activity than the native protein. Inclusion bodies, which commonly form when expressing proteins having disulfide bonds, and laborious in vitro refolding procedures can also be avoided by directed evolution.

According to the invention, proteins that are more easily expressed in facile gene expression systems can be obtained by using directed evolution to generate mutant polynucleotides in a library format for selection. General methods for generating libraries and isolating and identifying improved proteins (also described as “variants”) according to the invention using directed evolution are described briefly below and more extensively, for example, in U.S. Pat. Nos. 5,741,691 (90) and 5,811,238 (91). See also, International Applications WO 98/42832, WO 95/22625, WO 97/20078, WO 95/41653 and WO 98/27230 and U.S. Pat. Nos. 5,605,793 and 5,830,721 (23, 89, 92-97). It should be understood that any method for generating mutations in polynucleotide sequences to provide an evolved polynucleotide for use in expression systems can be employed. Proteins produced by directed evolution methods can then be screened for improved expression, folding, secretion, and function according to conventional methods.

Any source of nucleic acid, in purified form can be utilized as the starting nucleic acid. Thus the process may employ DNA or RNA including messenger RNA, which DNA or RNA may be single or double stranded. In addition, a DNA-RNA hybrid which contains one strand of each may be utilized. The nucleic acid sequence may be of various lengths depending on~the size of the nucleic acid sequence to be mutated. Preferably the specific nucleic acid sequence is from 50 to 50,000 base pairs. It is contemplated that entire vectors containing the nucleic acid encoding the protein of interest may be used in the methods of this invention.

Any specific nucleic acid sequence can be used to produce the population of mutants by the present process. An initial population of the specific nucleic acid sequences having mutations may be created by a number of different known methods, some of which are set forth below.

Error-prone polymerase chain reaction (33, 58, 59) and cassette mutagenesis (51-57), in which the specific region optimized is replaced with a synthetically mutagenized oligonucleotide can be employed in the invention, Error-prone PCR can be used to mutagenize a mixture of fragments of unknown sequences. These techniques can also be employed under low-fidelity polymerization conditions to introduce a low level of point mutations randomly over a long sequence, or to mutagenize a mixture of fragments of unknown sequence.

Oligonucleotide-directed mutagenesis, which replaces a short sequence with a synthetically mutagenized oligonucleotide may also be employed to generate evolved polynucleotides having improved expression.

Alternatively, nucleic acid or DNA shuffling, which, in one format, uses a method of in vitro or in vivo homologous recombination of pools of nucleic acid fragments or polynucleotides, can be employed to generate polynucleotide molecules having variant sequences of the invention.

Parallel PCR is another method that can be used to evolve polynucleotides for improved expression in conventional expression systems, which uses a large number of different PCR reactions that occur in parallel in the same vessel, such that the product of one reaction primes the product of another reaction. Sequences can be randomly mutagenized at various levels by, e.g., random fragmentation and reassembly of the fragments by mutual priming. Site-specific mutations can be introduced into long sequences by random fragmentation of the template followed by reassembly of the fragments in the presence of mutagenic oligonucleotides.

A particularly useful application of parallel PCR, which can be used in the invention, is called sexual PCR. In this technique, parallel PCR is used to perform recombination on a pool of DNA sequences. Sexual PCR can also be used to construct libraries of chimaeras of genes from different species.

The polynucleotide sequences for use in the invention can also be altered by chemical mutagenesis. Chemical mutagens include, for example, sodium bisulfite, nitrous acid, hydroxylamine, hydrazine or formic acid. Other agents which are analogues of nucleotide precursors include nitrosoguanidine, 5-bromouracil, 2-aminopurine, or acridine. Generally, these agents are added to the PCR reaction in place of the nucleotide precursor thereby mutating the sequence. Intercalating agents such as proflavine, acriflavine, quinacrine and the like can also be used. Random mutagenesis of the polynucleotide sequence can also be achieved by irradiation with X-rays or ultraviolet light, or by subjecting the polynucleotide to propagation in a host (such as E. coli) that is deficient in the normal DNA damage repair function. Generally, plasmid DNA or DNA fragments so mutagenized are introduced into E. coli and propagated as a pool or library of mutant plasmids.

Alternatively a mixed population of specific nucleic acids may be found in nature in that they may consist of different alleles of the same gene or the same gene from different related species (i.e., cognate genes). Alternatively, they may be related DNA sequences found within one species, for example, the peroxidase class of genes. Once the mixed population of the specific nucleic acid sequences is generated, the polynucleotides can be used directly or inserted into an appropriate cloning vector, using techniques well-known in the art.

Once the evolved polynucleotide molecules are generated they can be cloned into a suitable vector selected by the skilled artisan according to methods well known in the art. If a mixed population of the specific nucleic acid sequence is cloned into a vector it can be clonally amplified by inserting each vector into a host cell and allowing the host cell to amplify the vector. The mixed population may be tested to identify the desired recombinant nucleic acid. The method of selection will depend on the recombinant nucleic acid desired. For example, in this invention a recombinant nucleic acid which encodes for a protein with improved folding properties can be determined by tests for functional activity of the protein and absence of inclusion body formation. Such tests are well known in the art.

Using the methods of directed evolution, the invention provides a novel means for producing properly folded, functional, and soluble proteins in conventional or facile expression systems such as E. coli or yeast. Conventional tests can be used to determine whether a protein of interest produced from an expression system has improved expression, folding and/or functional properties. For example, to determine whether a polynucleotide subjected to directed evolution and expressed in a foreign host cell produces a protein with improved folding, one skilled in the art can perform experiments designed to test the functional activity of the protein. Briefly, the evolved protein can be rapidly screened, and is readily isolated and purified from the expression system or media if secreted. It can then be subjected to assays designed to test functional activity of the particular protein in native form. Such experiments for various proteins are well known in the art, and are discussed in the Examples below.

In one embodiment, the invention contemplates the use of polynucleotides encoding for variants of heme-containing proteins. Thus, the invention employs directed evolution to generate novel peroxidase enzymes, such as HRP, which fold properly in the host cells (e.g. E. coli) used in the expression system, retain functional activity, and avoid the problems associated with inclusion body formation.

The invention can also be applied to select or optimize an expression system, including selection of host cells, promoters, and signal sequences. Expression conditions can also be optimized according to the invention.

The Examples below further describe the methods of the invention and, in particular, teach the use of directed evolution to generate variants of HRP which when expressed using conventional expression systems do not form inclusion bodies and retain functional activity. Ordinarily, the corresponding native proteins form inclusion bodies and show little retained functional activity after expression in conventional expression systems.

Examples of practicing the invention are provided. The Examples are understood to be exemplary only, and do not limit the scope of the invention or the appended claims. A person of ordinary skill in the art will appreciate that the invention can be practiced in many forms according to the claims and disclosures herein.

### EXAMPLE 1

### Functional Expression of Horseradish Peroxidase in E. coli and Yeast

There is growing interest in exploiting eukaryotic peroxidases for use as industrial biocatalysts. Protein engineering and directed evolution to improve specific properties, however, are complicated by the lack of facile recombinant expression systems. In an effort to develop a functional bacterial expression system suitable for large-volume screening of mutants of horseradish peroxidase (HRP), the present Example describes the development of a bacterial expression system for heme-associated proteins, such as horseradish peroxidase (HRP), by inserting a corresponding gene as a fusion to the signal peptide PelB. In addition, by subjecting these genes to directed evolution, heme-associated proteins fold more efficiently in E. coli and are rendered more resistant to heat (thermostable) and more resistant to inactivation by H₂O₂. This Example provides an approach for greatly facilitating efforts to “fine-tune” many enzymes that are promising industrial biocatalysts, but for which suitable bacterial or yeast expression systems are currently lacking because the proteins form inclusion bodies or are inefficiently secreted by the cells.

A. Cloning of HRP

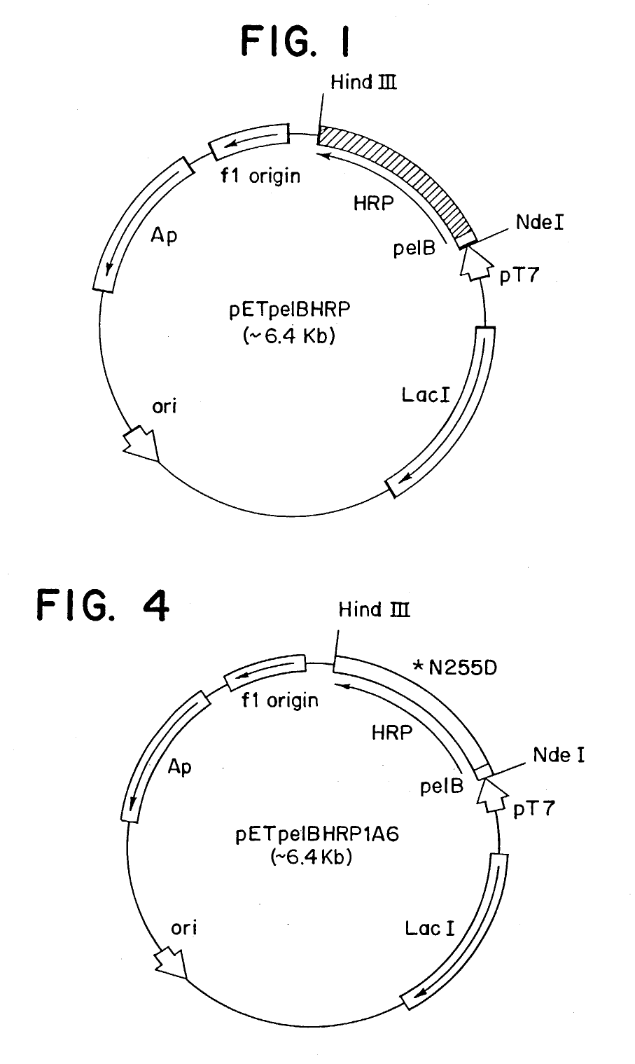

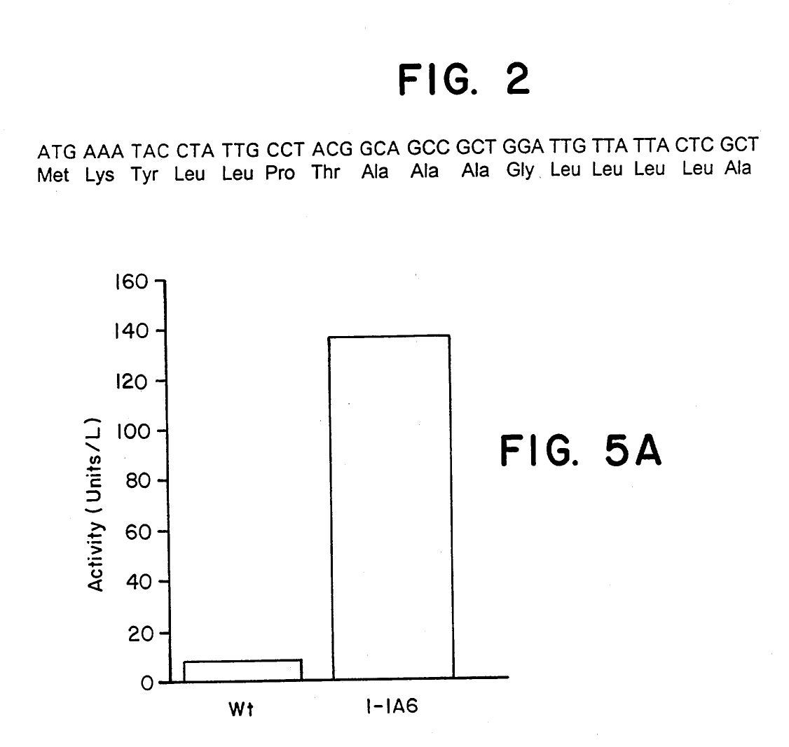

The HRP gene (with an extra methionine residue at the N-terminus) was cloned from the plasmid pBBG10 (British Biotechnologies, Ltd., Oxford, UK) by PCR techniques to introduce an Aat II site at the start codon and a Hind III site immediately downstream from the stop codon. This plasmid contains the synthetic horseradish peroxidase (HRP) gene described in Smith et al. (26), whose DNA sequence is based on a published amino acid sequence for the HRP protein (62). pBBG10 was made by inserting the HRP sequence between the HindIII and EcoR1 sites of the polylinker in the well-known plasmid pUC19. The PCR product obtained from this plasmid was digested with Aat II first, blunt-ended with t4 DNA polymerase, and then further restricted with Hind III. The digested product was ligated into pET-22b(+) (purchased from Novagen) treated with McsI and Hind III, to yield the vector pETpelBHRP. A map of this expression vector shown in FIG. 1. In this construct, the HRP gene was placed under the control of the T7 promoter and is fused in-frame to the pelB signal sequence (See [SEQ. ID NO.1 and SEQ. ID NO.2] and FIG. 2), which theoretically directs transport of proteins into the periplasmic space, that is, for delivery outside the cell cytoplasm (40). The ligation product was transformed into E. coli strain BL21(DE3) for expression of the protein in cells both with and without induction by 1 mM isopropyl-b-D-thiogalactopyranoside (IPTG).

In the cells that were induced with IPTG, no peroxidase activity above background was detected, for BL21(DE3) cells or pET-22b(+)-harboring BL21(DE3) cells, even though the level of HRP polypeptides accounted for over 20% of total cellular proteins. This was consistent with previous observations (25-27).

In the cells that were not induced with IPTG, clones were discovered that showed weak but measurable activity against azino-di-(ethylbenzthiazoline sulfonate (ABTS).

The T7 promoter in the pET-22b(+) vector is known to be leaky (44), and in theory it is therefore possible that some of the HRP polypeptide chains produced at this basal level were able to fold into the native form. Conversely, addition of IPTG leads to high-level HRP synthesis, which instead favors aggregation of chains and prevents their proper folding. Subsequently, random mutagenesis and screening were used to identify mutations that lead to higher expression of HRP activity.

Thus, one aspect of the invention includes the use of a promoter that can regulate production of small amounts of polypeptide under some conditions, and larger amounts under other conditions. For example, a “leaky” inducible promoters can be used. This type of promoter produces high levels of a particular protein or proteins in the presence of an inducer compound, and much lower levels in the absence of inducer. In some embodiments, a polypeptide can be over-expressed under certain conditions (e.g. in the presence of inducer) and under-expressed in other conditions (e.g. without inducer). Polypeptides that are inactive when expressed at normal levels or when over-expressed, but are active when under-expressed, are particularly suitable for use as parent polypeptides of the invention. Such expression-resistant polypeptides can be improved, using the methods of the invention, to provide functional, active expression at suitably high yields and activity levels.

B. Random Library Generation and Screening

One of the HRP clones that showed detectable peroxidase activity was used in the first generation of error-prone PCR mutagenesis. The random libraries were generated by a modification of the error-prone PCR protocol described above (33-35), in which 0.15 mM of MnCl₂ was used instead of 0.5 mM MnCl₂. This protocol incorporates both manganese ions and unbalanced nucleotides, and has been shown to generate both transitions and transversions and therefore a broader spectrum of amino acid changes (63).

Briefly, the PCR reaction solution contained 20 fmoles template, 30 pmoles of each of two primers, 7 mM MgCl₂, 50 mM KCl, 10 mM Tris-HCl (pH 8.3), 0.01% gelatin, 0.2 mM dGTP, 0.2 mM dATP, 1 mM dCTP, 1 mM dTTP, 0.15 mM MnCl₂, and 5 unit of Taq polymerase in a 100 μl volume. PCR reactions were performed in a MJ PTC-200 cycler (MJ Research, Mass.) for 30 cycles with the following parameters: 94° C. for 1 min, 50° C. for 1 min, and 72° C. for 1 min. The primers used were:

| | |
|---|---|
| 5′-TTATTGCTCAGCGGTGGCAGCAGC, and | [SEQ. ID NO.15] |
| 5′-AAGCGCTCATGAGCCCGAAGTGGC. | [SEQ ID NO:16] |

The PCR products were purified with a Promega Wizard PCR kit, and digested with Nde I and Hind III. The digestion products were subjected to gel-purification with a QIAEX II gel extraction kit, and the HRP fragments were ligated back into the similarly digested and gel-purified pET-22b(+) vector. Ligation mixtures were transformed in the BL21(DE3) cells by electroporation with a Gene Pulser II (Bio-Rad). Cell growth and expression was carried out in either 96-well or 384-well microplates in LB medium at 30° C. Peroxidase activity tests were performed with H2O₂ and ABTS (39).

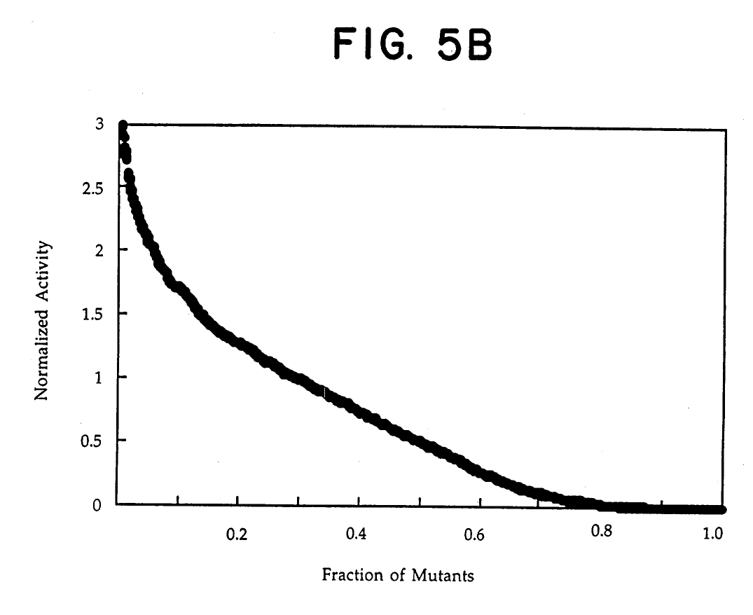

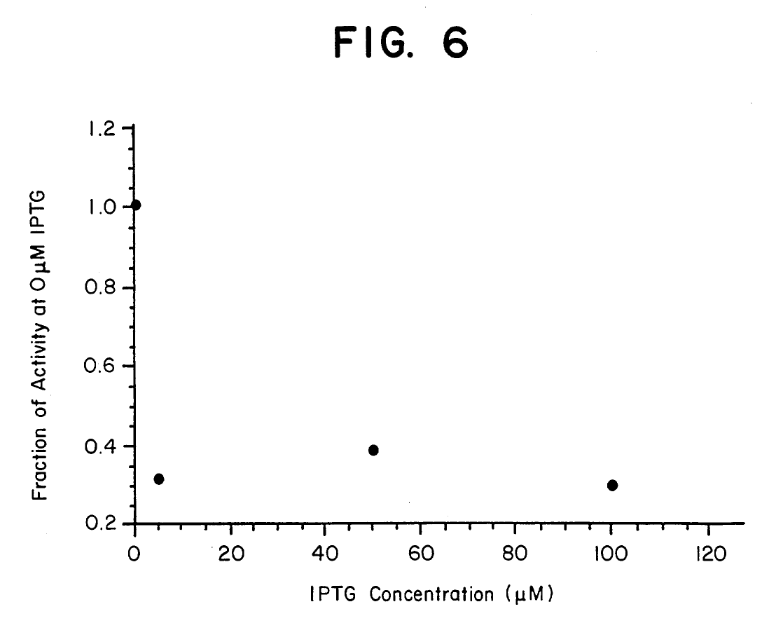

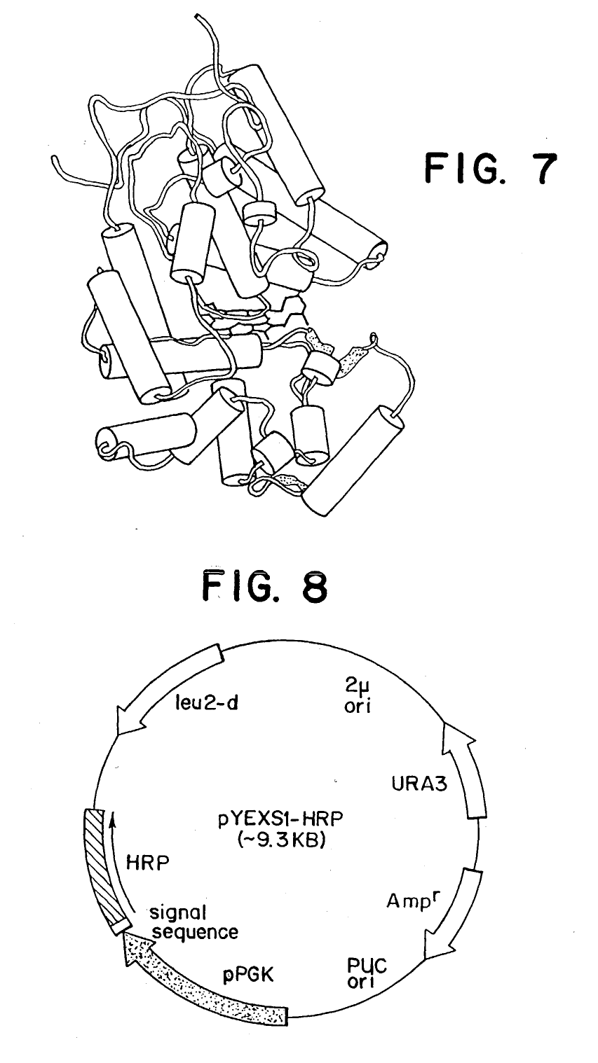

For each generation, typically 12,000-15,000 colonies were picked and screened in 96-well plates. This number represents an exhaustive search of all accessible single mutants, with a probability of 95% for any mutant to be sampled at least once (38). Colonies were either picked manually, or using an automated colony picker at Caltech, Q-bot (Genetix, UK). Of the 12,000 colonies that were screened (no IPTG added), a clone designated HRP1A6 showed 10-14 fold higher peroxidase activity than the parent clone. (FIGS. 5A and 5B). This clone also showed markedly decreased activity when as little as 5 μM of IPTG was added. FIG. 6. Sigma reports that 1 mg of highly purified HRP from horseradish has a total activity of 1,000 units, as determined by the ABTS assay. Other workers reported similar results (26). Based on this data, the concentration of active HRP was estimated to be about 100 μg/L. HRP1A6 shows a total activity of greater than 100 units/L. This compares favorably with the yield obtained from refolding of aggregated HRP chains in vitro (26). The level of expression for the HRP1A6 clone is also similar to that for bovine pancreatic trypsin inhibitor (BPTI) in E. coli (45), an unglycosylated protein with three disulfide bonds. Greater than 95% of the HRP activity was found in the LB culture medium as judged by the ABTS activity.

The HRP1A6 clone remained stable for up to a week at 4° C. IPTG was omitted in all HRP expression experiments, unless otherwise specified. Peroxidase activity tests for HRP were performed with a classical peroxidase assay, ABTS and hydrogen peroxide (39). Fifteen μl of cell suspension was mixed with 140 μl of ABTS/H₂O₂ (2.9 mM ABTS, 0.5 mM H202, pH 4.5) in microplates, and the activity was determined with a SpectraMax plate reader (Molecular Devices, Sunnyvale, Calif.) at 25° C. A unit of HRP is defined as the amount of enzyme that oxidizes 1 μmole of ABTS per min at the assay conditions.

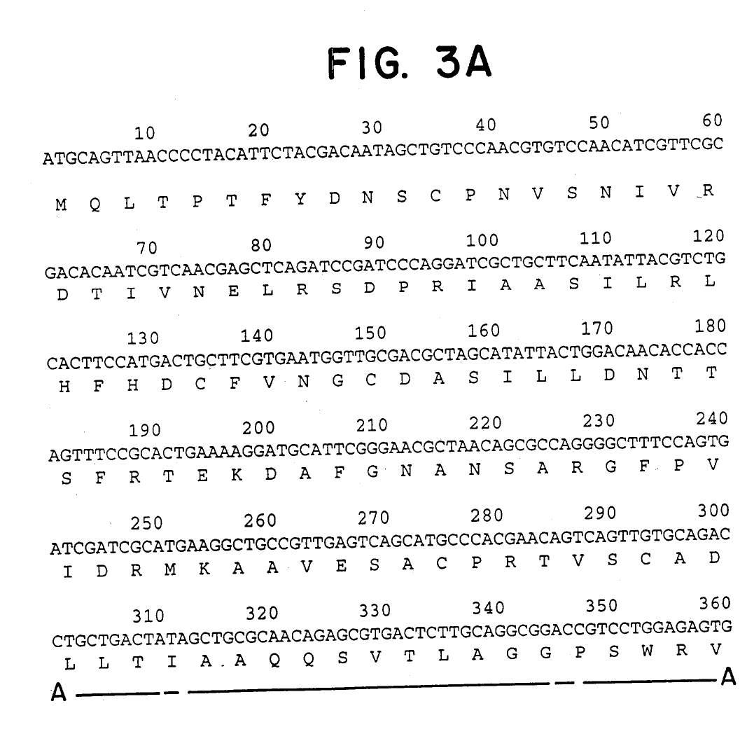

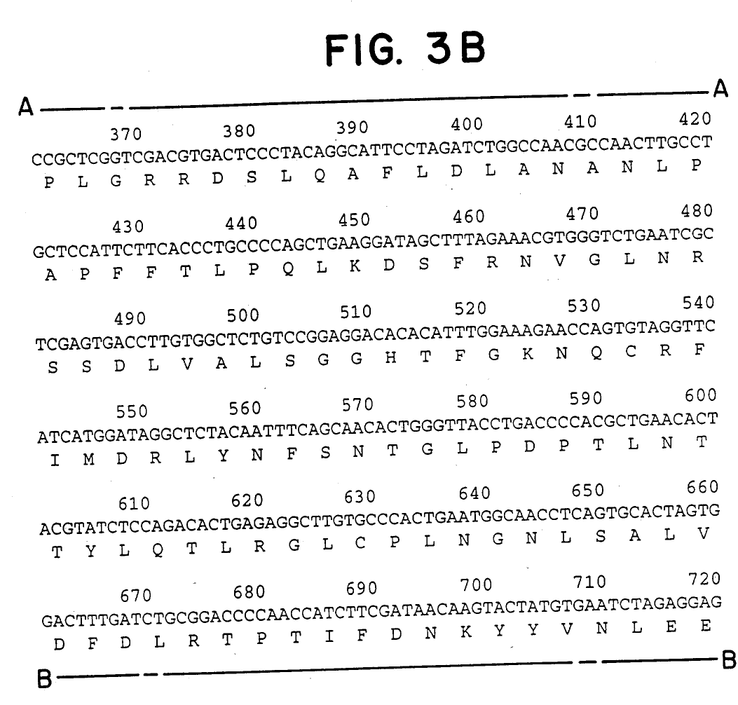

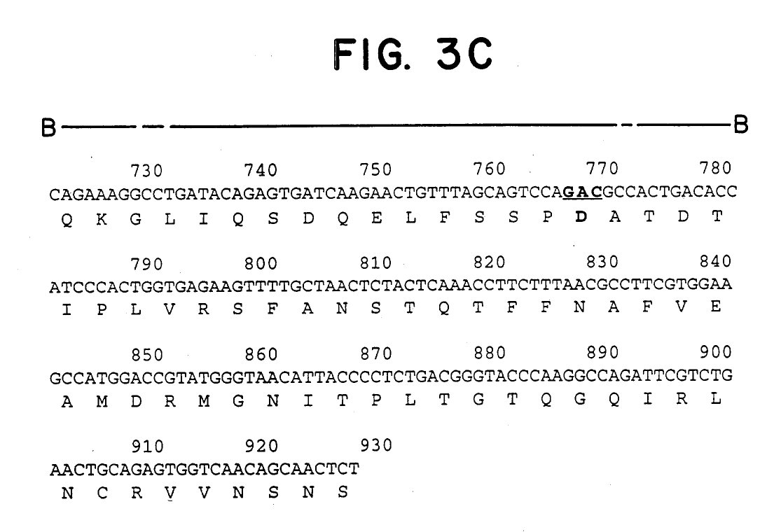

HRP1A6 demonstrated higher expression and/or peroxidase activity than wild-type HRP. Sequencing of the HRP1A6 gene revealed that its amino acid sequence is identical to that of wild-type HRP. This indicates that the HRP1A6 gene contains a mutation outside of the protein encoding region that results in an enhanced peroxidase activity of this clone. The sequence of recombinant wild-type HRP1A6) is shown in FIG. 3 [SEQ ID NO:3]. A map of a plasmid pETpelBHRP1A6 containing the HRP1A6 gene is shown in FIG. 4.

C. Functional Expression of HRP in Yeast

The native HRP protein contains four disulfide bonds, and E. coli has only a limited capability to support disulfide formation. In theory, these well-conserved disulfides in HRP (and other plant peroxidases) are likely to be important for the structural integrity of the protein, and may not be replaceable by mutations elsewhere. Yeast has a much greater ability to support the formation of disulfide bonds. Thus, yeast can be used as suitable expression host, in place of E. coli, particularly if it is desired to relieve the apparent limitation on the folding of HRP imposed by any constraints on disulfide formation in E. coli. For example, S. cerevisiae can be used as a host for the expression of mutant HRP genes and proteins.

The recombinant wild-type HRP gene HRP1A6 was cloned into the secretion vector pYEX-S1 obtained from Clontech (Palo Alto, Calif.) (60), yielding pYEXS1-HRP (FIG. 8). This vector utilizes the constitutive phosphoglycerate kinase promoter and a secretion signal peptide from Kluveromyces lactis. The plasmid was first propagated in E. coli, and then transformed into S. cerevisiae strain BJ5465, obtained from the Yeast Genetic Stock Center (YGSC), University of California, Berkeley using the LiAc method as described (61). BJ5465 is protease deficient, and has been found to be generally suitable for secretion. A first generation of error-prone PCR of HRP in yeast was performed. Among the first 7,400 mutants screened, four variants showed 400% higher activity than HRP1A6 in yeast. Additional details and results are given in Example 2.

### EXAMPLE 2

### Functional Expression of HRP in Yeast through Directed Evolution

This example describes the use of directed evolution to further improve the functional expression of HRP. As explained in Example 1, the horseradish peroxidase gene HRP1A6 was isolated. Since HRP contains four well-conserved disulfides, and E. coli has only limited ability to support disulfide bond formation, the further improvement in bacterial expression of HRP in E. coli may be constrained by correct pairing of disulfide-containing cysteines. Yeast cells, for example S. cerevisiae, have much greater ability to support the formation of disulfide bonds, and may be better able to accommodate disulfide bonds in peroxidase enzymes. In theory, these well-conserved disulfides in HRP (and other plant peroxidases) are likely to be important for the structural integrity of the protein, and may not be replaceable by mutations elsewhere. Thus, yeast can be used as suitable expression host, in place of E. coli, particularly if it is desired to relieve the apparent limitation on the folding of HRP imposed by any constraints on disulfide formation in E. coli.

Accordingly, S. cerevisiae was chosen as an alternative host for the expression of HRP. S. cerevisiae is both a micro-organism and a eukaryote, and possesses much of the eukaryotic protein post-translational and secretory machinery, such as ER and Golgi that catalyze the formation of disulfide bonds and glycosylate polypeptides. Genetic manipulation techniques (in particular gene transformation) are also readily available. A drawback is that yeast naturally secrete few proteins. Moreover, yeast glycosylation differs significantly from that in higher eukaryotic organisms, which might present problems for secretion of glycoproteins (17). Nonetheless, several proteins have been efficiently secreted from yeast (17). Stategically, the experiments of this example take advantage of the capacity of yeast to catalyze the formation of disulfide bonds while fine-tuning the glycosylation factor through the process of directed evolution.

A. Construction of Yeast Expression System for HRP.

HRP1A6 from Example 1 was cloned into the yeast secretion vector pYEX-S1 obtained from Clontech (Palo Alto, Calif.) (60), yielding pYEXS1-HRP (FIG. 8). This vector utilizes the constitutive phosphoglycerate kinase promoter and a secretion signal peptide from K. lactis. pYEX-S1 was digested with SacI, and then blunt-ended with T4 DNA polymerase. The mature HRP1A6 gene was cloned from pETpelBHRP1A6 by PCR techniques using the proofreading polymerase pfu (Stratagene, Calif.) that generate blunt-end products. The PCR fragments were then ligated into the restricted and blunt-ended pYEX-S1, and transformed into E. coli DH5α cells. A number of colonies were picked and grown in liquid culture, from which plasmids were prepared. Sequencing identified several positive colonies with the correct orientation and the correct nucleotide sequence. This yeast expression vector is generally referred to hereinafter as pYEXS1-HRP (FIG. 8). In this construct, the HRP gene was placed directly downstream of the secretion signal peptide from K. lactis, and the expression is under the control of the constitutive phosphoglycerate kinase promoter. The vector also carries the E. coli Amp resistance gene as well as the yeast selectable markers leu2-d and URA3 (60).

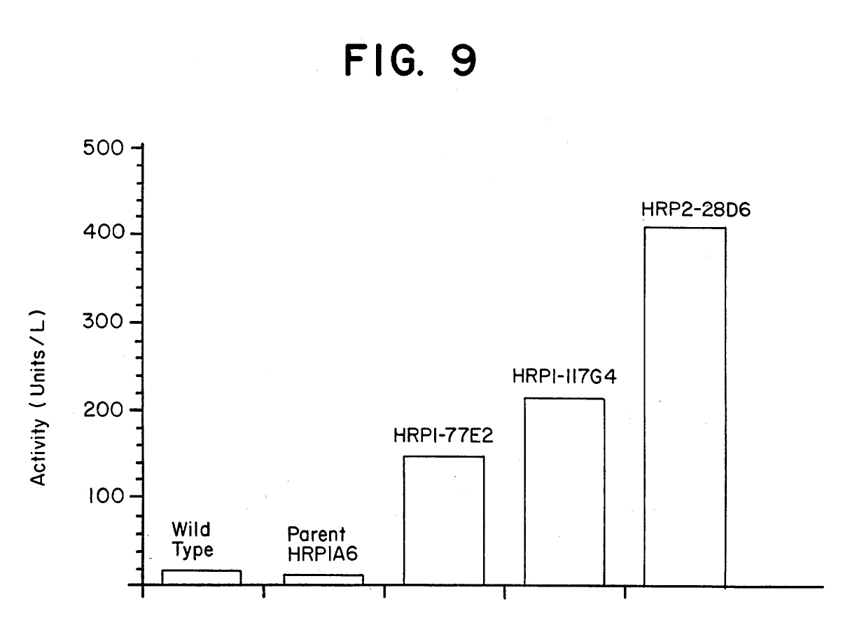

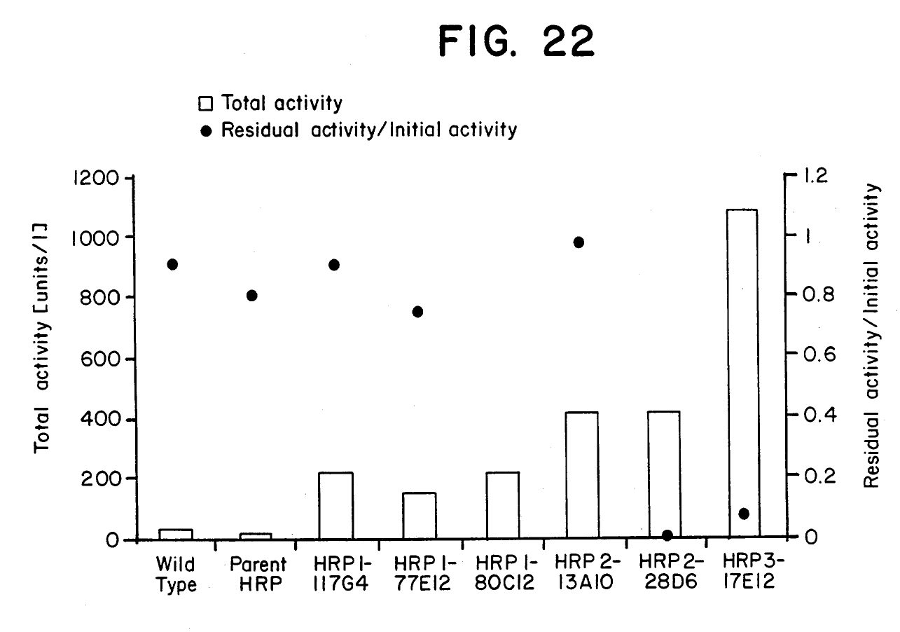

For expression experiments, the plasmid was first propagated in E. coli strain DH5α, and then transformed into S. cerevisiae strain BJ5465, obtained from the Yeast Genetic Stock Center (YGSC; University of California, Berkeley), using a LiAc method that utilizes single strand DNA as described by Gietz et al. (61). BJ5465 is protease deficient and generally suitable for secretion (17). Following transformation, cells were plated on YNB selective medium supplemented with 20 μg/ml leucine, 20 μg/ml histidine, 20 μg/ml adenine and 20 μg/ml tryptophan. Colonies were picked, and grown in 96-well microplates in YEPD medium at 30° C. in an air-circulating incubator for 2 days and 16 hours. HRP activity tests were performed with a classical peroxidase assay, ABTS and hydrogen peroxide (39). The activity obtained from yeast for HRP1A6 was only about 1/10, about 25 units/L (FIGS. 9 and 22), of that from E. coli, and actually slightly lower than obtained for the wild-type in this construct.

B. First Generation HRP Mutagenesis in Yeast for Improving Expression.

A first generation of error-prone PCR of HRP1A6 in yeast was aimed at improving the expression level. An error-prone PCR protocol incorporating both unbalanced nucleotide concentrations and manganese ions as described previously (33, 34) was used. This protocol was shown to generate roughly random mutations, allowing for sampling of a broader spectrum of amino acid residue changes. The manganese ion concentration used was 100 μM, which generated an error rate of approximately 1-2 mutations per gene on average (35). The PCR products were purified with a Promega Wizard PCR kit, digested with Sac I and Bam HI (thus the first 27 amino acid residues of HRP were left unmodified). The digestion products were then subjected to gel-purification with a QIAEX II gel extraction kit, and the HRP fragments were ligated back into the similarly digested and gel-purified PEXS1-HRP1A6. Ligation mixtures were transformed in HB101 cells by electroporation with a Gene Pluser II (Bio-Rad). Colonies were scratched from the E. coli plates and resuspended in LB medium, from which plasiids were prepared. Then the plasmids were transformed into yeast and yeast colonies were obtained and grown as described above.

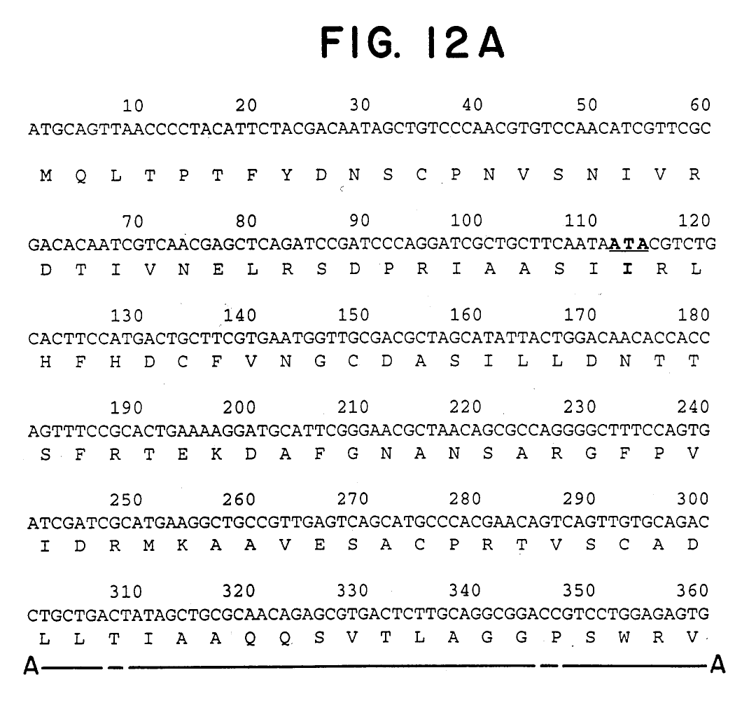

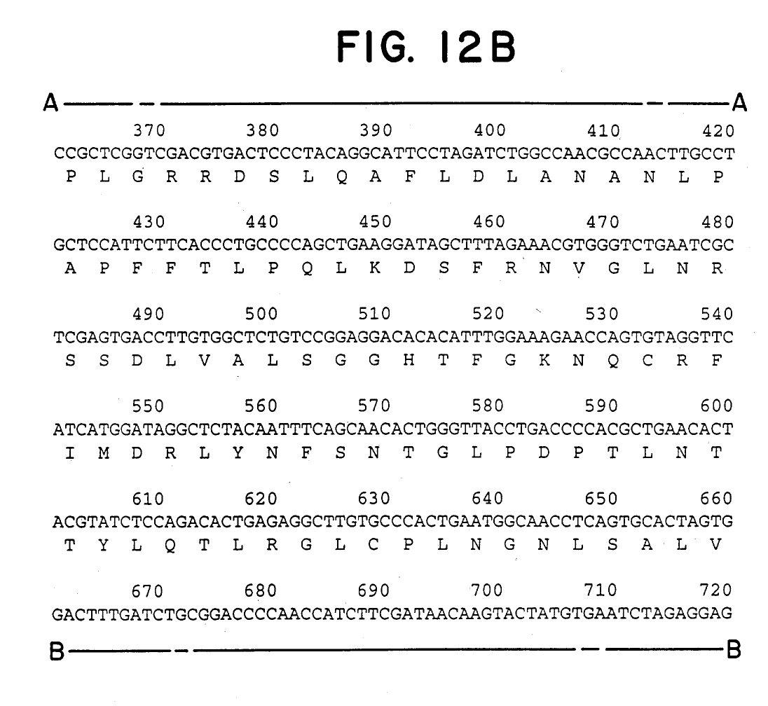

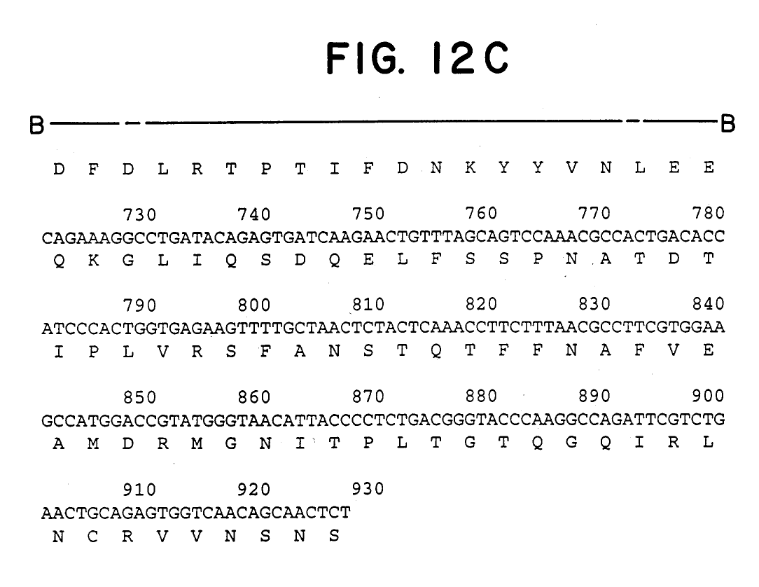

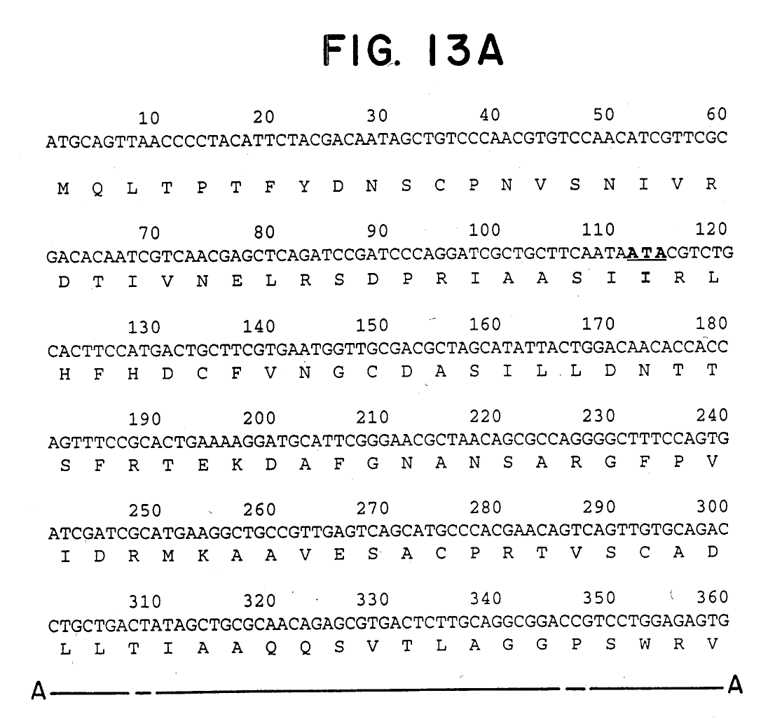

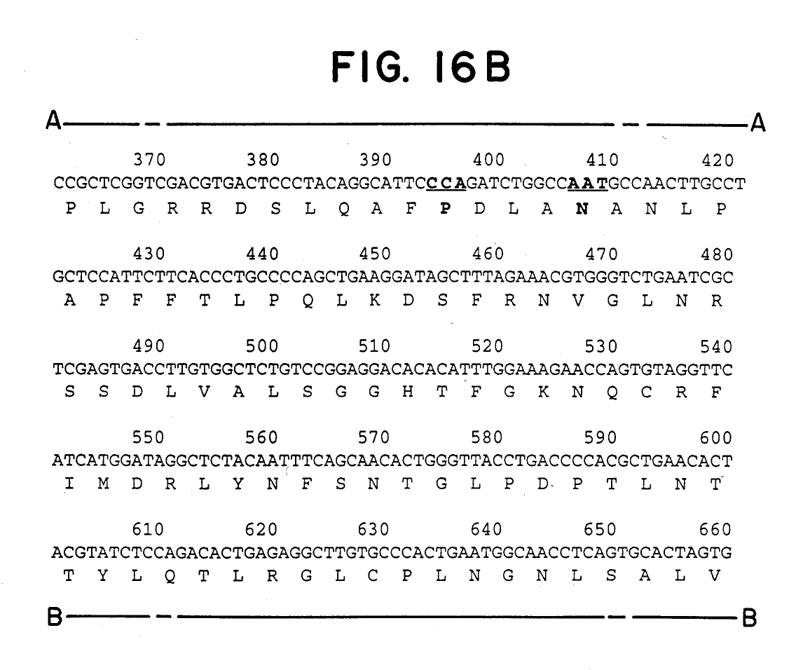

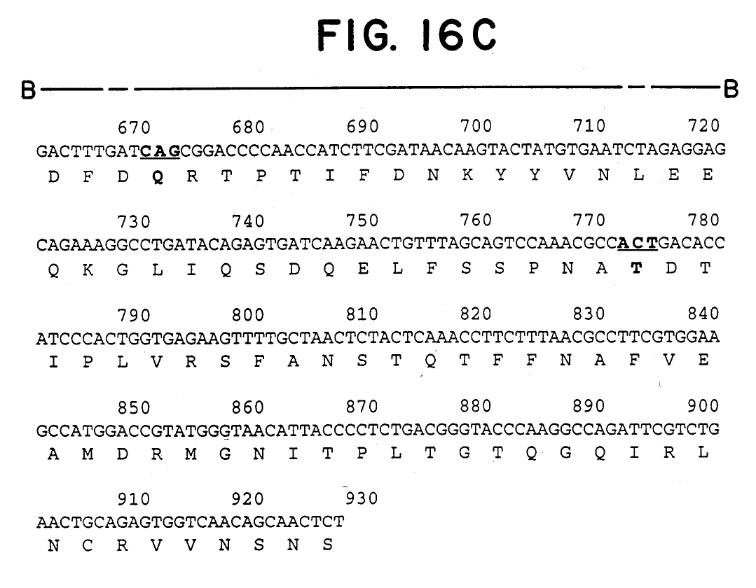

A total of about 14,000 colonies were picked and screened for this generation, which represented an exhaustive search of all accessible single mutants, and a probability of 95% for any mutant to be sampled at least once (38). Of these colonies, a number of mutants showed significantly higher activity than the parent (HRP1A6) in yeast. Two exemplary improved mutants are designated HRP1-117G4 [SEQ ID NO:12 and SEQ ID NO:13] and HRP1-77E2 [SEQ ID NO:5 and SEQ ID NO:6]. HRP1-117G4 gave a 16-fold higher activity than the parent, or a total activity of about 220 units/L (FIG. 9). HRP1-77E2 showed a total activity of about 147 units/L. Both of these were higher than the highest level obtained from E. coli. See also FIG. 12 (HRP1-77E2) and FIG. 16 (HRP1-117G4).

C. Second Generation of HRP Mutagenesis in Yeast for Improving Expression.

The second generation of error-prone PCR used HRP1-117G4 as the parent. For this generation, a higher concentration of manganese ion was used to increase the mutation rate. This change was made based on the following considerations. Since screening can only handle a library of about 10⁴ to 10⁵ mutants at the present time, the rate of mutagenesis has been conservatively limited to creating predominately single mutants in the past (28). In this example, the fraction of clones more active than the parent for a given generation remains relatively constant with the error-rate up to 6 mutations per gene. The advantage of using higher error rates is that it would allow neutral mutations to exist along with beneficial mutations isolated through screening. These accrued neutral mutations may become useful in subsequent generations by either providing a bridge for generating new types of mutations, or by synergetic interactions with newly created mutations. The manganese ion concentration used in this generation was 350 μM, which generated an error rate of approximately 4-5 mutations per gene on average (35).

Additionally, a prescreening of the colonies using nitrocellulose membranes was performed. This was possible because the higher error-rate significantly reduce the number of colonies that showed similar or higher activity than the parent. The procedures were as follows. Colonies were first replicated from the master plates onto nitrocellulose membranes and grown on YEPD plates at 30° C. for one day and 6 hours. The membranes were then retrieved from the plates and immersed in a mixture of TMB (tetramethylbenzidine) and H₂O₂. The colonies with the brightest color were identified, and corresponding mother colonies were picked and grown from the master plates. For this generation, about 120,000 colonies were screened (about 5,000 were actually picked and grown), and the mutant HRP2-28D6 was obtained. It showed an activity 85% higher than its parent, HRP1-117G4, or a total activity of 410 units/L (FIG. 9).

D. First Generation of HRP Mutagenesis in Yeast for Improving Stability

One generation of random mutagenesis of HRP for improving thermostability and resistance towards H₂O₂ was carried out using HRP1-77E2 as the parent. The random mutagenesis (with 100 μM manganese) and cell growth was essentially performed as described above (with no prescreening). Thermostability tests were performed with a MJ PTC-200 cycler (MJ Research, MA) at 73° C. with an incubation time of 10 min. H₂O₂ resistance tests were separately performed in 25 mM H₂O₂ at room temperature and a pre-incubation time of 30 min., followed by ABTS screening in 25 mM H₂O₂. Mutants that were more thermostable or chemically stable (H₂O₂ resistant) than the parent were further characterized at various temperatures (for thermostability) or H₂O₂ concentrations (for H₂O₂ stability).

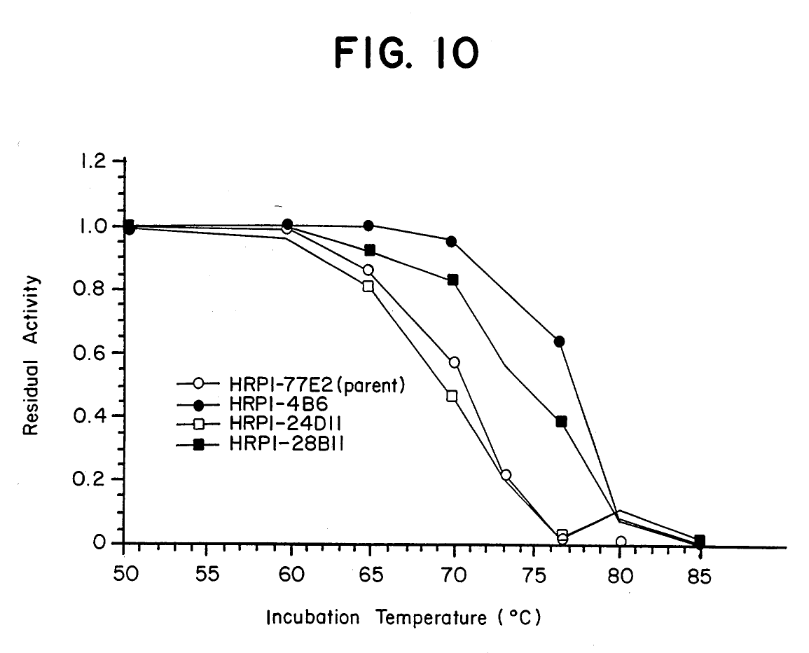

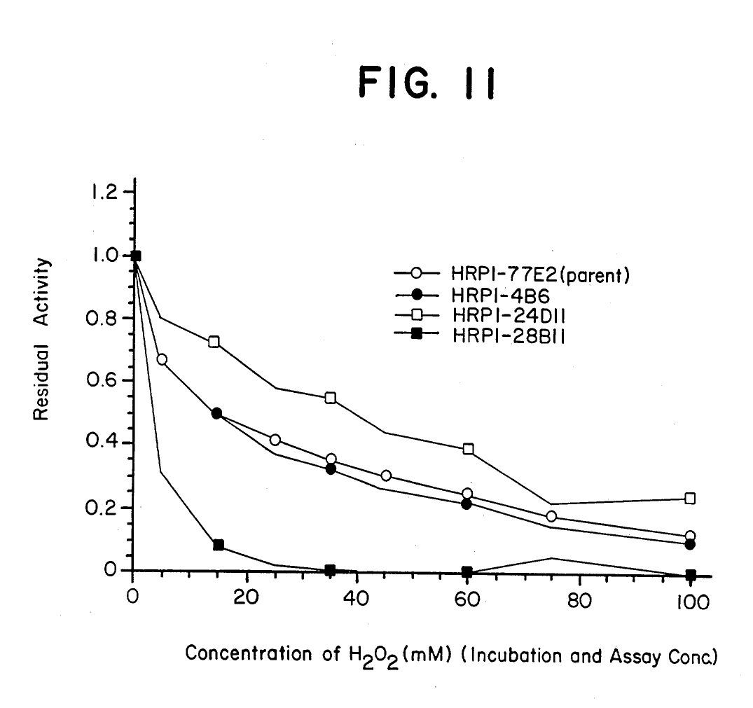

Out of 3,000 colonies screened, one thermostable mutant (HRP1-4B6) showed a T_{½} of over 6° C. higher than that of the parent (T_{½} is the transition midpoint of the HRP inactivation curve as a function of temperature) (FIG. 10). Another mutant, HRP1-28B11 also showed some improvement in thermostability. The mutant HRP1-24D11 was not markedly more thermostable than its parent HRP1-77E2, but was more resistant to H₂O₂ degradation. (A feedback mechanism common to HRP enzymes is that they are degraded by H₂O₂, which is a reactant in the enzymatic reactions that HRP facilitates.) The HRP1-24D11 mutant retained about 60% of activity after incubation with 25 mM H₂O₂ for 30 min, while the parent exhibited a 42% residual activity under the same conditions (FIG. 11).

E. Sequencing Data

Sequencing revealed that HRP1-77E2 carries the mutation L37I (TTA to ATA). This residue is part of the helix 2, and is near the heme pocket (47). See, FIG. 12, [SEQ ID NO:5] and [SEQ ID NO:6].

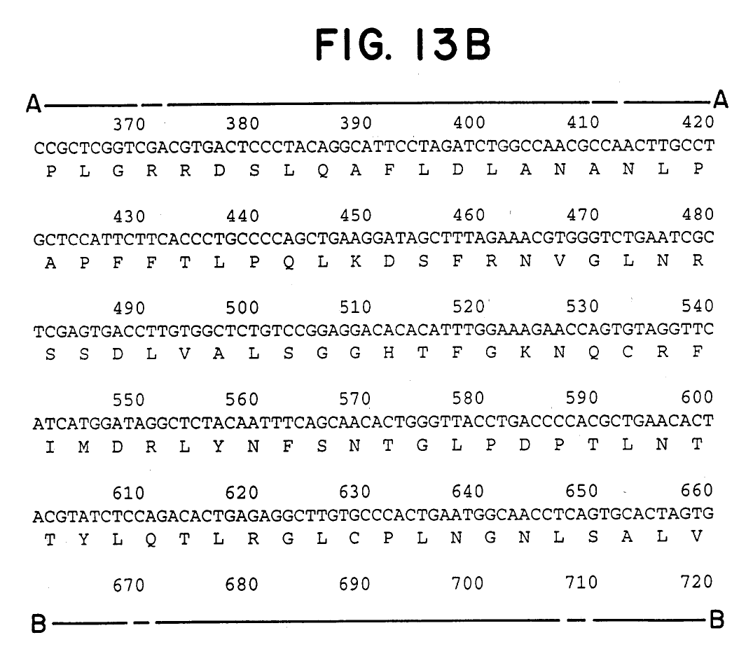

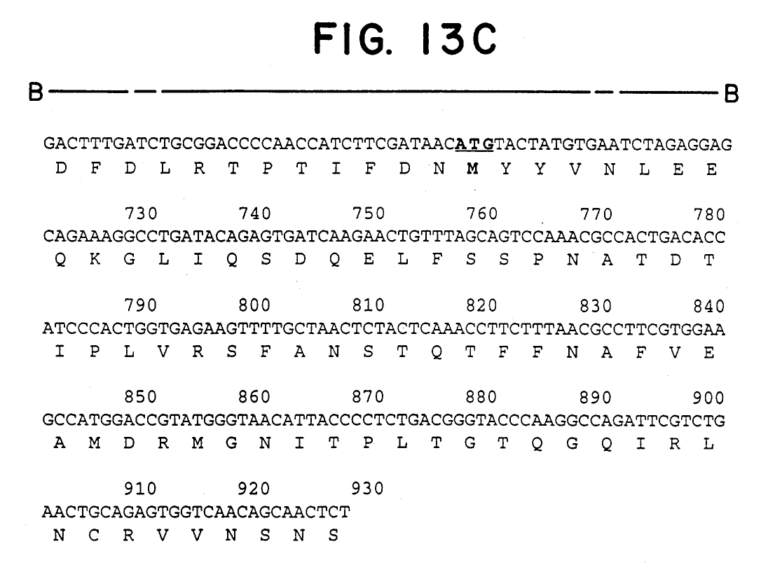

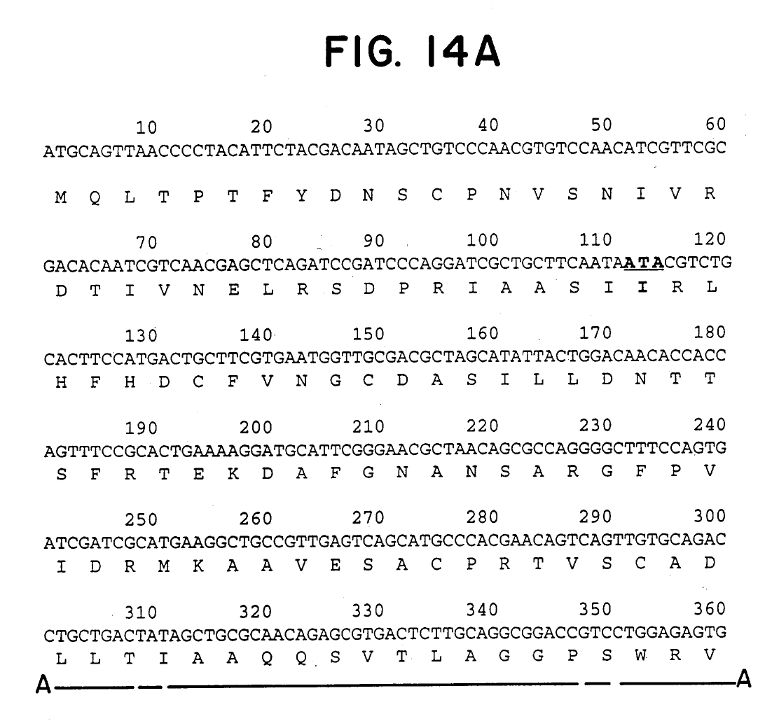

The mutant HRP1-4B6 carries K232M (AAG to ATG) in addition to L37I. This residue is part of the helix 14, and is exposed to solvent on the surface. See, FIG. 13, [SEQ ID NO:7] and [SEQ ID NO:8].

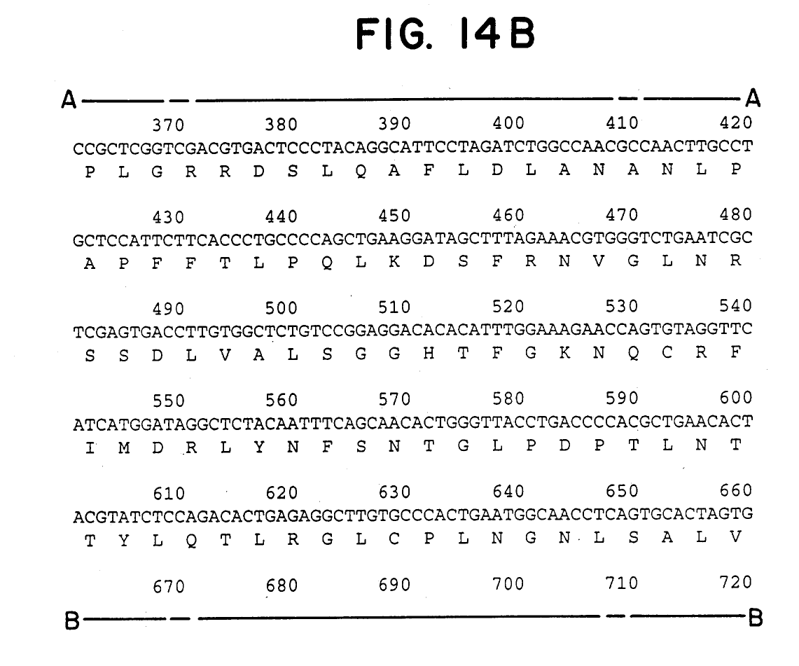

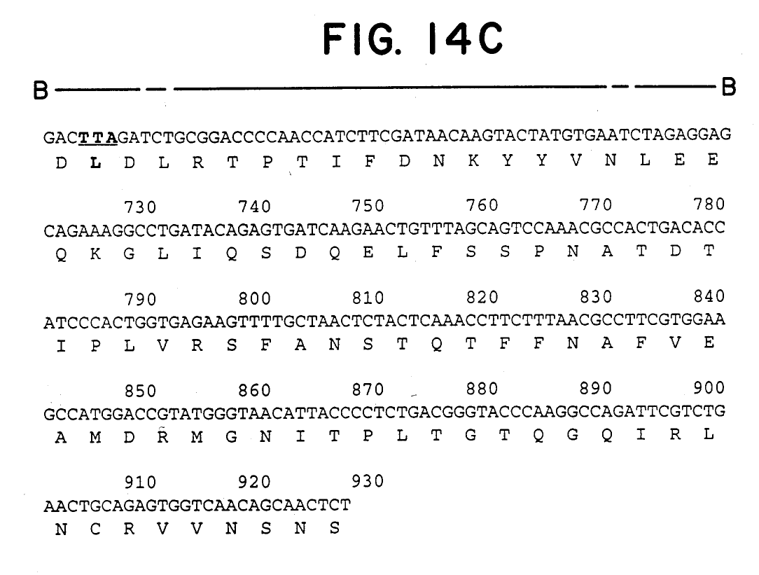

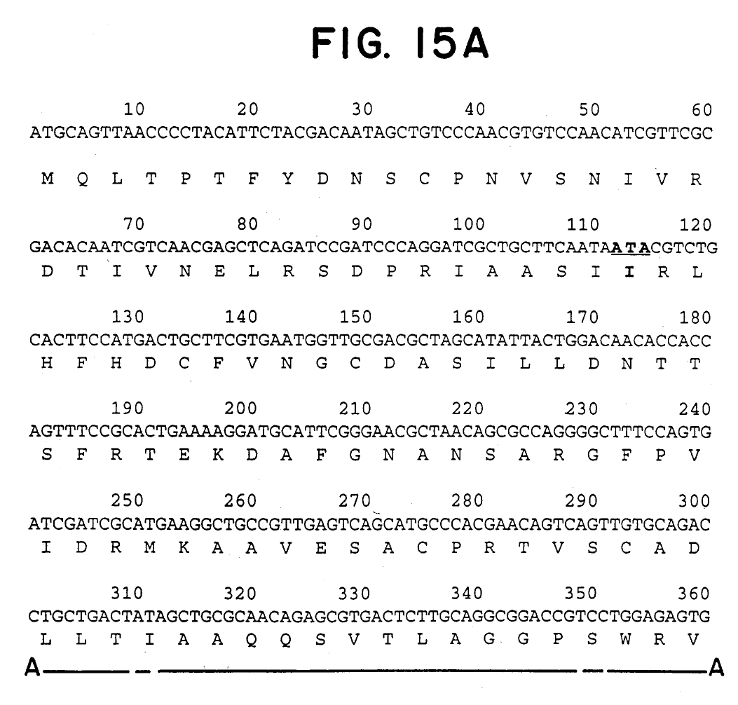

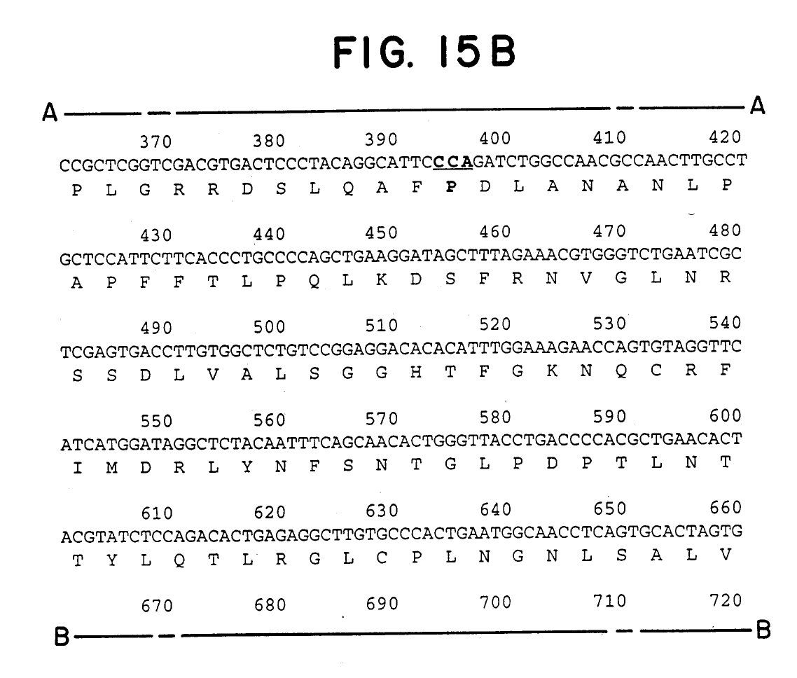

HRP1-28B 11, the mutant with thermostability between HRP1-77E2 and HRP1-4B6 has the mutation F221 L (TTT to TTA) in addition to L37I. This residue is in a structural loop and part of the substrate access channel (47). See, FIG. 14, [SEQ ID NO:9] and [SEQ ID NO:10].

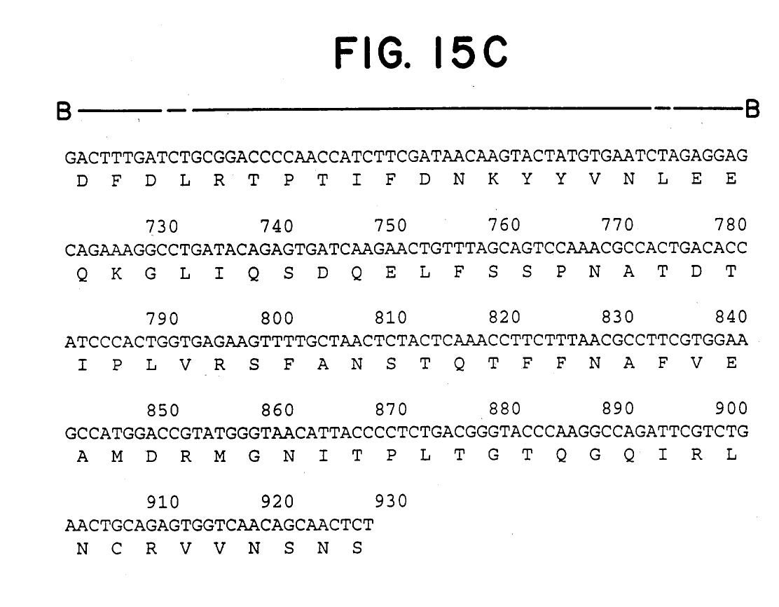

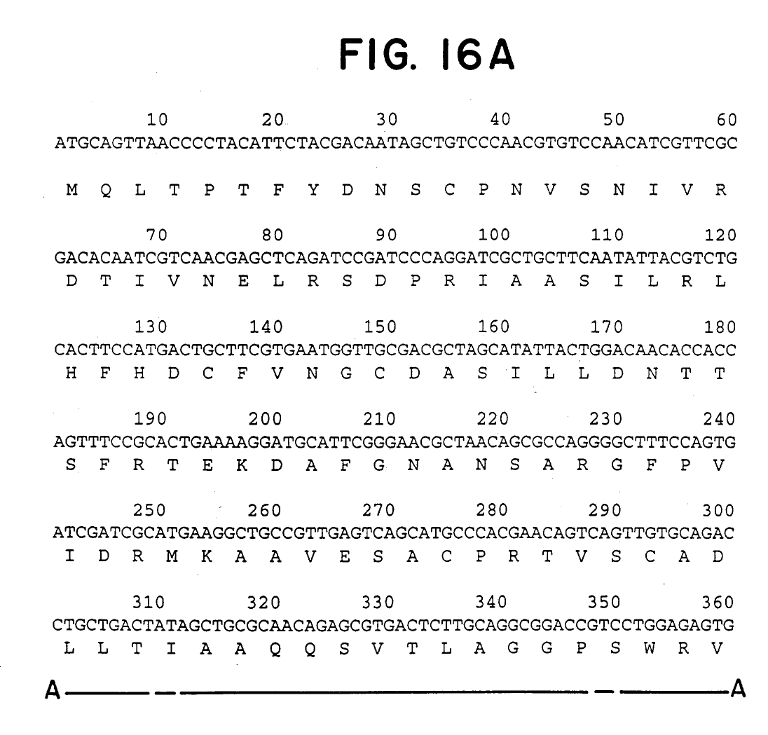

The mutant HRP1-24D11 contains the mutation L131P (CTA to CCA) in addition to L37I. This residue is at the tip of the helix 7, and is on the surface. See, FIG. 15, [SEQ ID NO:11] and [SEQ ID NO:12].

The mutant HRP1-117G4, a preferred mutant from the first generation in terms of total activity, contains four mutations with respect to its parent: (1) L131P (CTA to CCA); (2) L223Q (CTG to CAG); with silent mutations (3) at N135 (AAC to AAT) and (4) T257 (ACT to ACA). For the mutation L223Q, this amino acid residue is in a loop and is exposed to solvent. See, FIG. 16, [SEQ ID NO:13] and [SEQ ID NO:14].

### EXAMPLE 3

### Expression and Secretion of CCP in E. coli

A. Construction of Expression Vector for CCP.

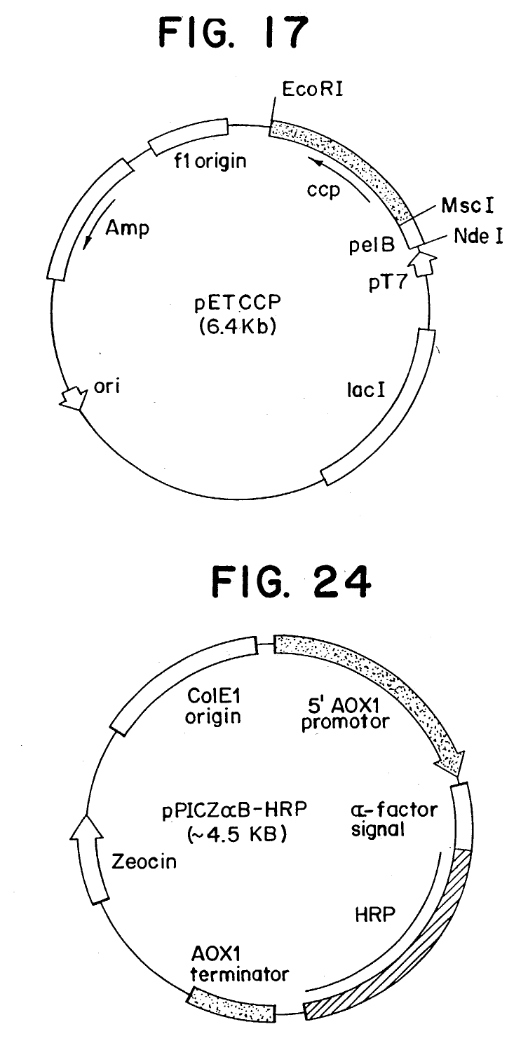

The S. cerevisiae cytochrome c peroxidase (CCP) gene from pT7CCP (29,30), donated by Dr. Dave Goodin, The Scripps Research Institute, La Jolla, Calif.) was recloned by PCR techniques to introduce an Msc I site at the start codon and a Hind III site immediately downstream from the stop codon. The PCR product was restricted with Msc I and Hind III, and then ligated into similarly digested pET-22b(+), yielding pETCCP (FIG. 17). The pT7CCP carries a gene for CCP in which the N-terminal sequence has been modified to code for amino acids Met-Lys-Thr, as described in Goodin et al. (30) and Fitzgerald et al. (29). Thus, in this construct, the CCP gene was placed under the control of the T7 promoter, and was fused in-frame to the pelB signal sequence for periplasmic localization.

B. Expression of CCP.

Expression experiments of CCP in E coli BL21(DE3) were carried out in LB medium containing 100 μg/ml ampicillin. Cells were grown at 37° C. to an A₆₀₀ of 0.7-0.8, at which time IPTG was added to a final concentration of 1 mM to induce the synthesis of CCP from the T7 promoter. Growth was continued at 30° C. for an additional 20 hours, and cells and supernatant were harvested by centrifugation.

CCP is known to fold correctly inside E. coli. Surprisingly, greater than 95% of the CCP protein was found in the LB culture medium at high levels (approximately 100 mg/liter, as assessed by SDS-PAGE). The protein was active towards ABTS, showing that the secreted CCP is folded and contains the required ferric heme.

Having thus described exemplary embodiments of the invention, it should be noted by those skilled in the art that the within disclosures are exemplary only and that various other alternatives, adaptations, and modifications may be made within the scope of the invention. For example, it will be understood by practitioners that the steps of any method of the invention can generally be performed in any order, including simultaneously or contemporaneously, unless a particular order is expressly required, or is necessarily inherent or implicit in order to practice the invention. Accordingly, the invention is not limited to any specific embodiments or illustrations herein. The invention is defined according to the appended claims, and is limited only according to the claims.

### EXAMPLE 4

### Directed Evolution and Sequences of Additional HRP Mutants in Yeast

Libraries of HRP mutants were constructed by error-prone PCR as described above. Two primers flanking the HRP gene were used in the PCR reactions:

| | |
|---|---|
| 5′-CAGTTAACCCCTACATTC-3′ and | [SEQ ID NO:25] |
| 5′-TGATGCTGTCGCCGAAGAAG-3′. | [SEQ ID NO:26] |

Thermal cycling parameters were 95° C. for 2 min, 94° C. for 1 min, 50° C. for 1 min, and 72° C. for 1 min (30 cycles) and 72° C. for 7 min.

The PCR products were purified and digested with Sac I and Bam HI (leaving the first 27 amino acid residues of HRP unmodified). The digestion products were then subjected to gel purification, and the HRP fragments were ligated back into the similarly digested and gel-purified pYEXS1-HRP1A6. Ligation mixtures were transformed into E. coli HB101 cells by electroporation with a Gene Pulser II from Bio-Rad (Hercules, Calif.) and selected on LB medium supplemented with 100 μg/ml ampicillin. Colonies were directly harvested together from LB plates and mixed. This plasmid DNA was subsequently used for transformation into S. cerevisiae BJ 5465 as described above.

Single colonies were picked from YNB plates and grown at 30° C. for 64 h in 96-well plates containing non-selective YEPD medium (1% yeast extract, 1% peptone, 2% glucose) in an incubator. Microtiter plates were then centrifuged at 1,500 g for 10 min, and 10 μl of the supernatant in each well was transferred to a new microplate with a Beckman 96-channel pipetting station (Multimek, Beckman, Fullerton, Calif.) and assayed for total HRP activity. Standard deviations for this measurement in 96-well plates on wild type clones did not exceed 10% (including pipetting errors, which contribute about 2%). Mutants showing the highest total HRP activity were retrieved from the microplates and re-grown in YNB selective medium. Plasmids were extracted from the cells with a Zymo yeast plasmid miniprep kit and returned to E. coli XL10-Gold for further propagation and preparative isolation.

In the 2nd and 3rd generations, HRP-expressing yeast clones were pre-screened as follows. Colonies on YNB plates were replicated onto pure nitrocellulose membranes and incubated on fresh YEPD agar at 30° C. for 34 hr. Membranes were then immersed in 100 ml of TMB substrate solution (0.8 mM TMB, 2.9 mM H₂O₂, and 0.12 % (W/V) dextran sulfate as enhancer) for 5 min to generate color. Yeast clones exhibiting bright green color were traced back to the master YNB selective plates and picked and grown in YEPD for further screening in plates, as described above.

To follow expression over time, S. cerevisiae expressing wild type and mutants HRP 2-13A10 and HRP 3-17E12 were grown in 100 ml YEPD medium in shake flasks. A 5% inoculum pregrown in YNB selective medium was used. Samples were taken at various times and assayed for HRP activity as described below.

HRP Activity: Peroxidase activity was measured using ABTS and hydrogen peroxide (Shindler et al., 1976). Cell suspensions (10 μl or 15 μl) or purified protein samples (1-6 ng) were mixed with 140 μl (or 150 μl) of ABTS/H₂O₂ (0.5 mM ABTS and 2.9 mM H₂O₂, pH 4.5) in a 96-well plate, and the increase of absorbance at 405 nm (ε of oxidized ABTS is 34,700 cm⁻¹M⁻¹) was determined with a SpectraMax plate reader (Molecular Devices, Sunnyvale, Calif.) at 25° C. The guaiacol assay was performed with 1 mM H₂O₂ and 5 mM guaiacol in 50 mM sodium phosphate buffer (pH 7.0). The increase in absorbance at 470nm was followed (ε of oxidized product at 470 nm is 26,000 cm⁻¹M⁻¹) after addition of the yeast culture supernatant (20 μl) or purified protein (15-30 ng). One unit is defined as the amount of enzyme that oxidizes 1 μmole of substrate per min under the assay conditions.

Stability of expressed mutants was determined after incubation at 50° C. for 10 min in NaOAc buffer (50 mM, pH 4.5) and is given as the ratio of residual activity and initial activity.

Deglycosylation of HRP: Purified protein samples were deglycosylated using EndH (New England BioLabs). The protein was denatured (0.5% SDS, 1% β-mercaptoethanol) and incubated with EndH for 1 h at 37° C.

A. First Generation Mutant HRP1-80C12

Using HRP1A6 (Example 1) as a parent, a first generation of error-prone PCR in yeast, according to the methods in Example 2, produced a mutant designated HRP1-80C12. (Plasmid pYEXS1-HRP1A6 was the parent gene.) HRP1-80C12 demonstrated HRP activity of213 units/L.

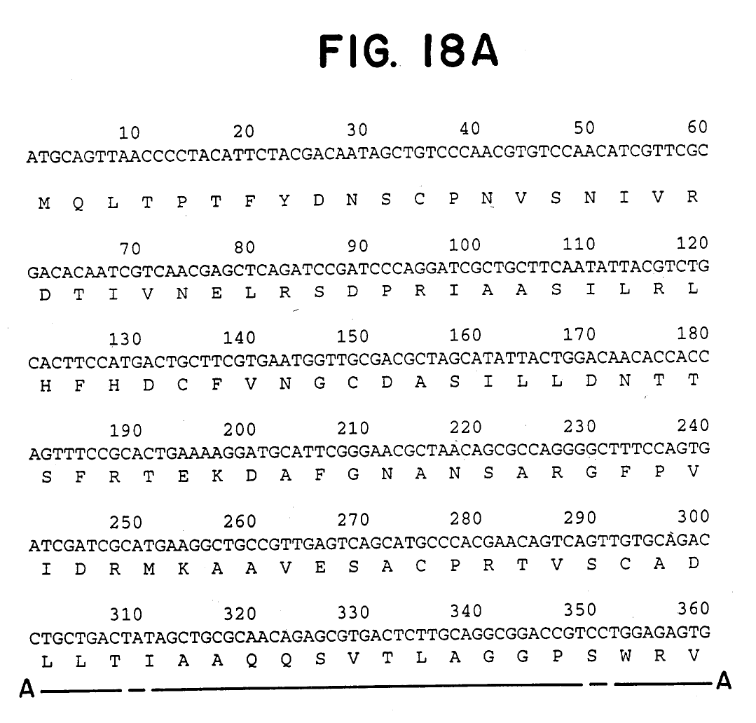

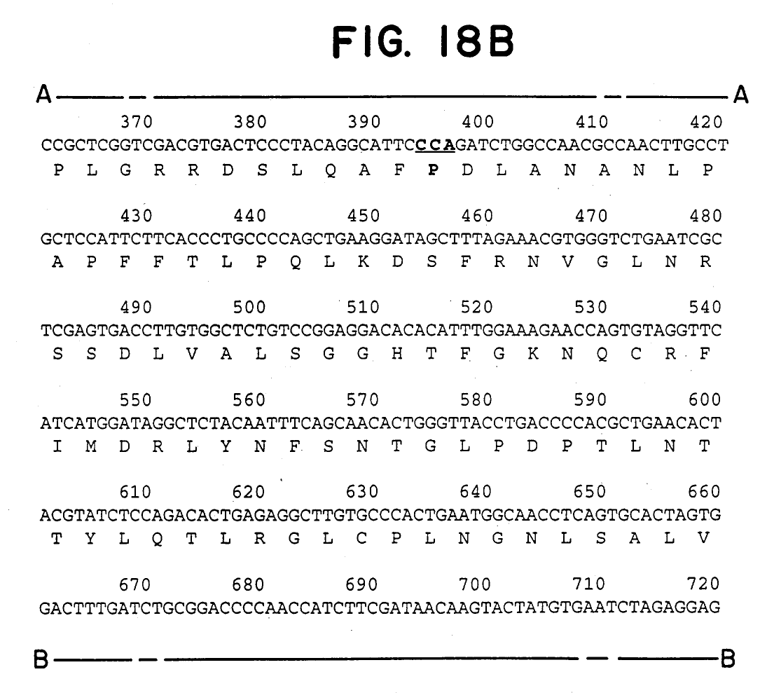

This is similar to the activity of HRP1-117G4 (about 220 units/L) and HRP1-77E2 (about 147 units/L) discussed above. All three mutants showed higher activity than the highest activity obtained from E. coli, about 140 units/L. FIG. 18 shows a nucleotide and amino acid sequence encoding mutant HRP1-80C12. ([SEQ ID NO:17 and SEQ ID NO:18]).

B. Second Generation Mutant HRP2-28D6

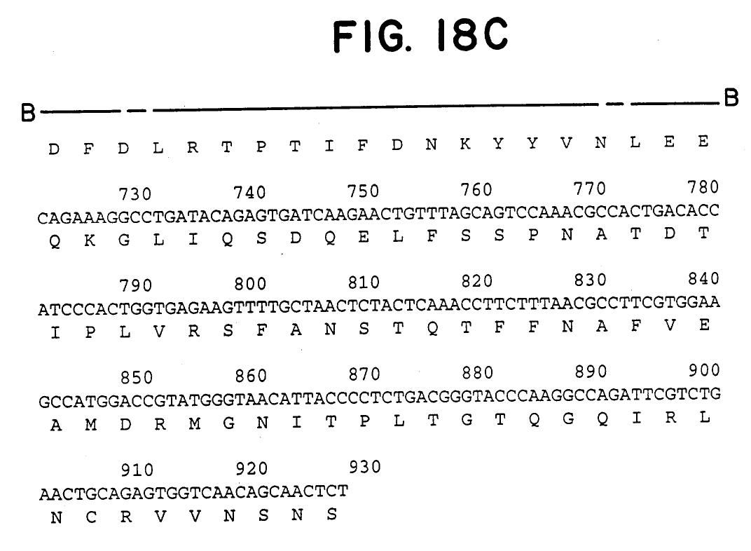

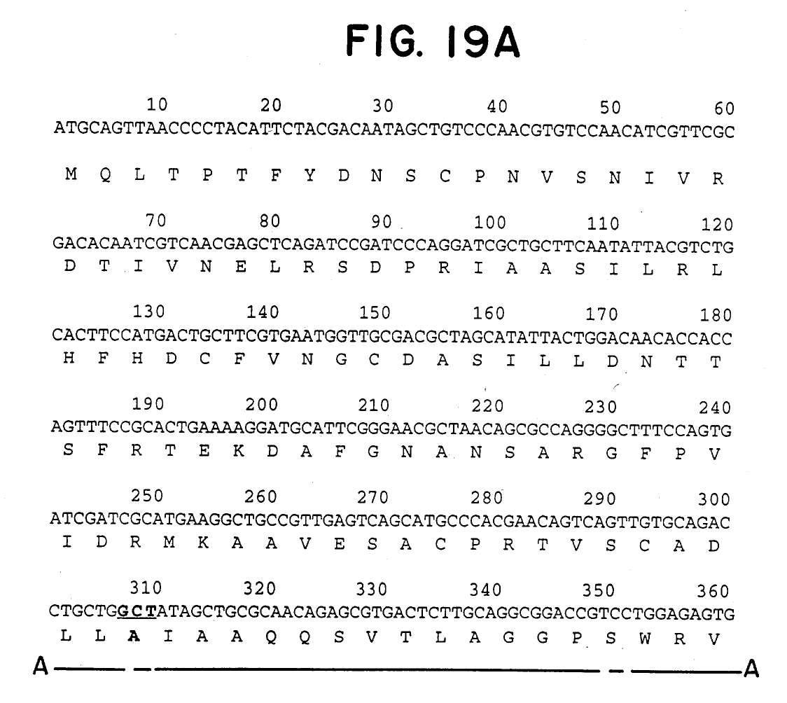

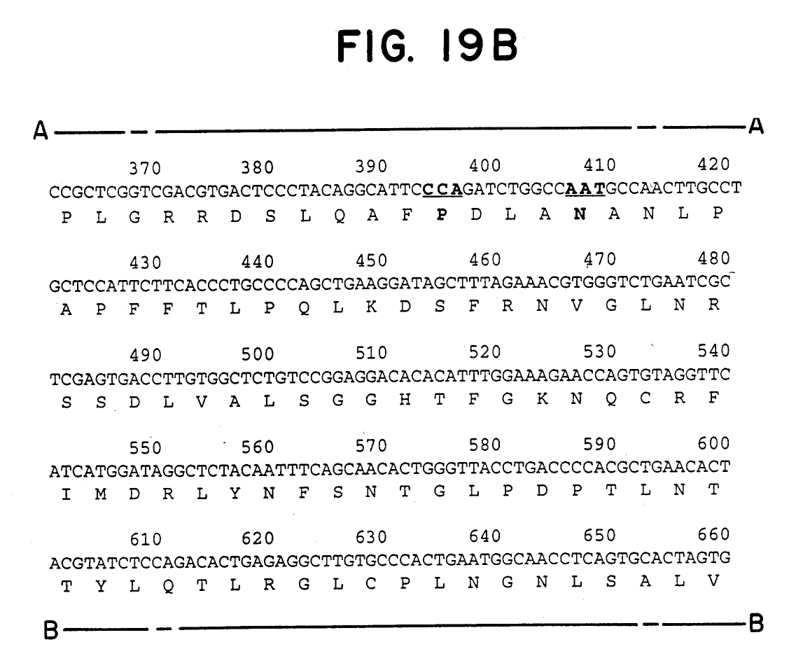

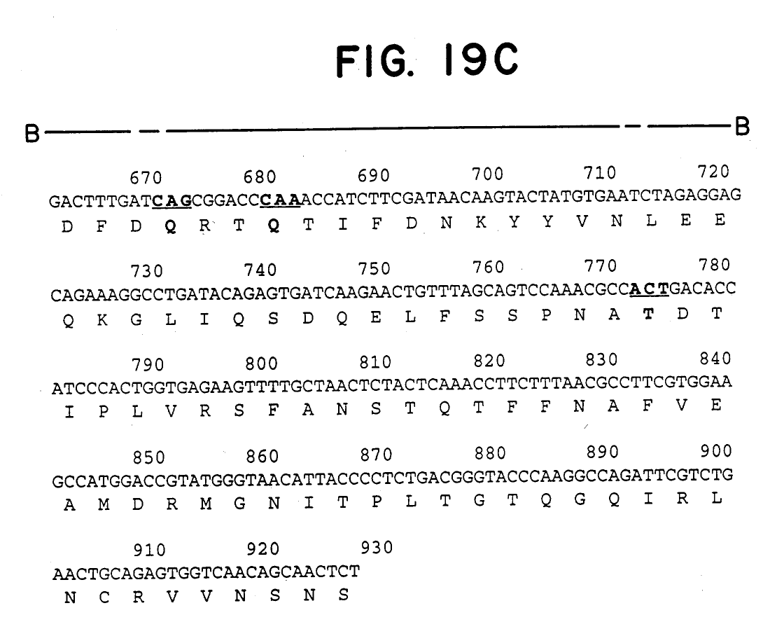

A nucleotide and amino acid sequence for second generation mutant HRP2-28D6 (Example 2) was obtained, and is shown in FIG. 19 (SEQ ID NO:19 and SEQ ID NO:20).

C. Second Generation Mutant HRP2-13A10

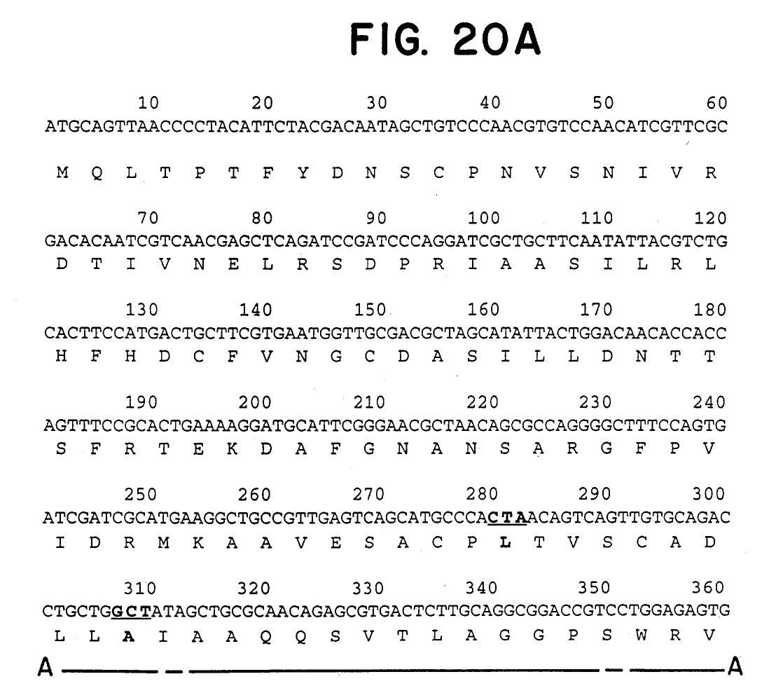

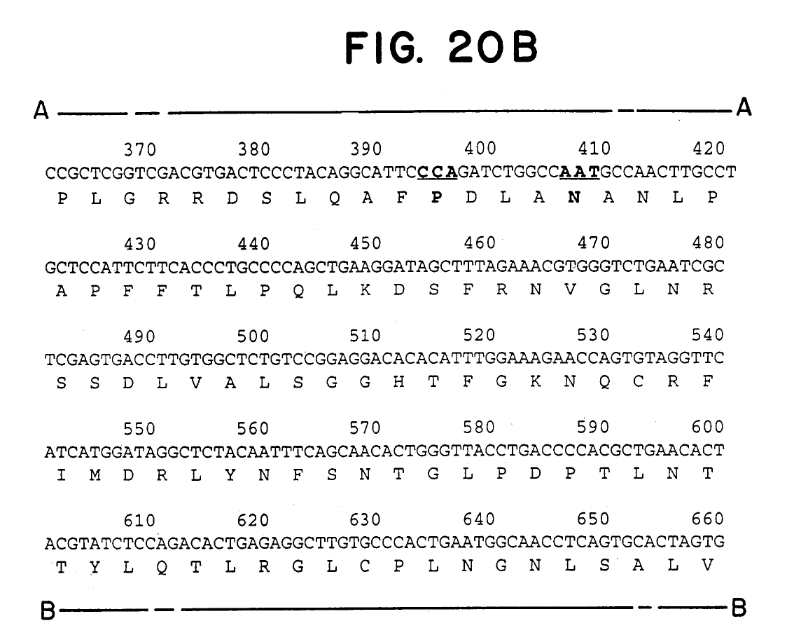

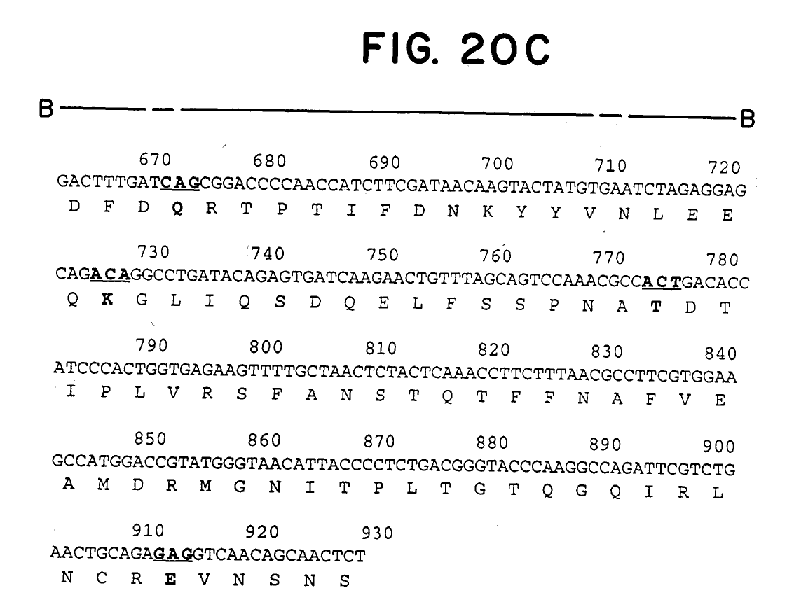

HRP1-1 17G4 was used as the parent for another round of error-prone PCR according to Example 2, except that a manganese ion concentration of 0.35 mM was used. This high error rate significantly reduced the fraction of colonies with activities comparable to wild type. In order to avoid screening large numbers of inactive clones, active clones were first identified using a facile pre-screening scheme with nitrocellulose membranes. Tetramethylbenzidine (TMB) membrane substrate was used to prevent diffusion of the colored assay products during the prescreening reactions. A good correlation between activity on ABTS and against TMB was observed for HRP mutants in solution (R=0.92). Colonies showing the brightest color reactions were picked from the master YNB plates for quantitative analysis of HRP activity in YEPD Medium. This round of error-prone PCR yielded mutant HRP2-13A10. Like mutant HRP2-28D6, HRP2-13A10 demonstrated a total activity of 410 units/L (1.8-fold higher than its parent). A nucleotide and amino acid sequence for second generation mutant HRP2-13A10 was obtained, and is provided in FIG. 20 (SEQ ID NO:21 and SEQ ID NO:22).

D. Third Generation Mutant HRP3-17E12

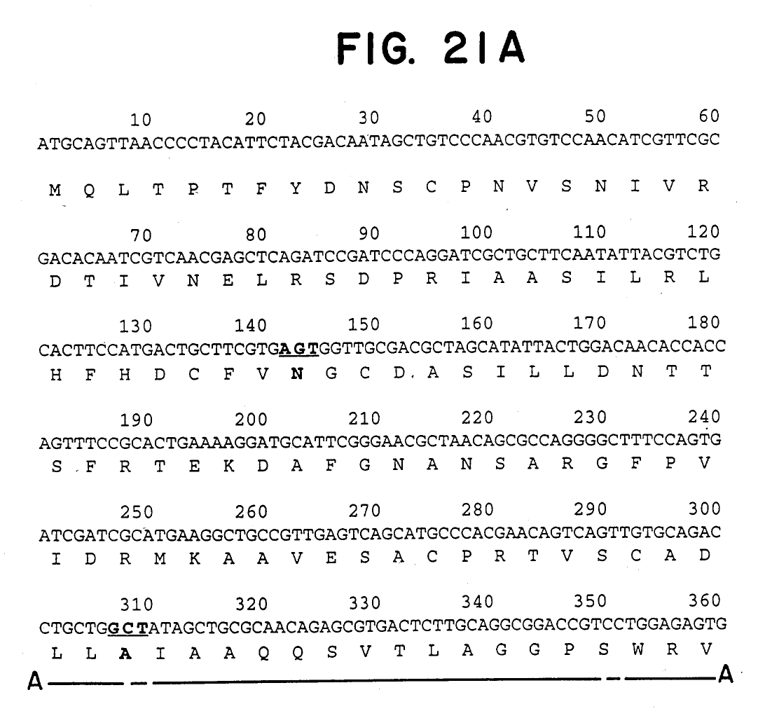

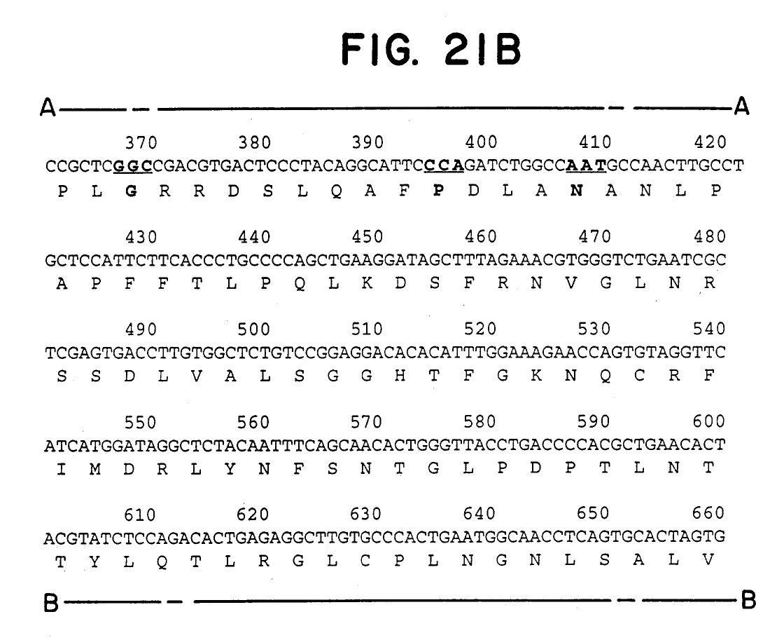

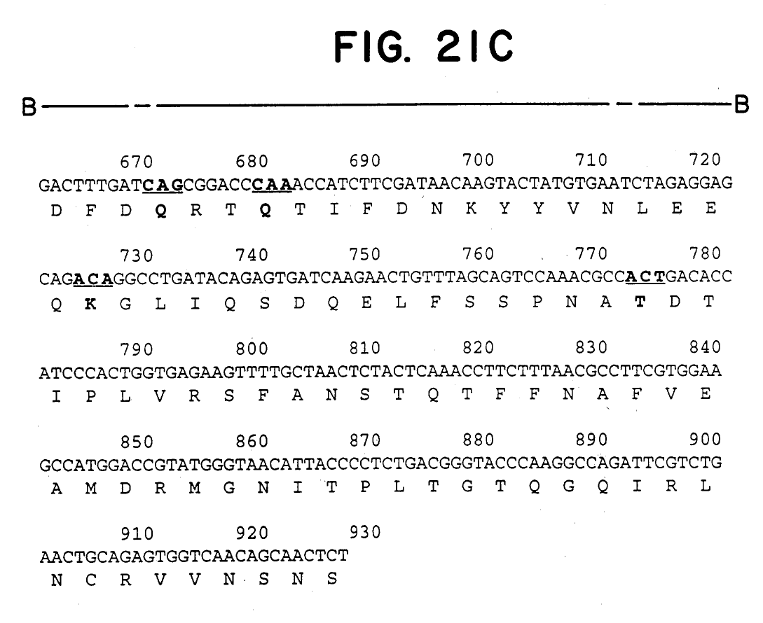

A third round of random mutagenesis was carried out using the methods of Example 2C and conditions similar to those for second generation mutagenesis, except that a manganese ion concentration of 0.35 mM was used (as in Example 4C) . HRP2-28D6 was the parent. For this generation, 90,000 colonies were prescreened, and 3,000 were picked and grown. From the resulting clones, a particularly active mutant was identified. This mutant, designated HRP3-17E12, produced 1080 units/L, an increase in HRP activity of 1.6-fold over parent HRP2-28D6, 83-fold over the starting mutant, HRP1A6, and 40-fold over wild type. A nucleotide and amino acid sequence for second generation mutant HRP3-17E12 was obtained, and is provided in FIG. 21 (SEQ ID NO:23 and SEQ. ID NO. 24).

E. Results of Sequeticing Representative Highly Active Mutants

Sequencing revealed that HRP1-117G4, the most active mutant from the first generation of these experiments in S. cerevisiae, has a total of four mutations: two non-synonymous mutations (L131P and L223Q ) and two synonymous mutations at N135 (AAC→AAT) and at T257 (ACT→ACA) (FIG. 16). The substitution L131P occurs at the tip of helix D′ and is on the surface (47). The L223Q mutation is in the loop connecting helix G and a β-sheet, and is exposed to the solvent. In addition, HRP1-80C12 contains the L131P substitution also found in HRP1-77G4. HRP1-77E2 has a different substitution, L37I. This residue is part of helix B and is in the heme pocket. It is presumably accessible to solvent and is adjacent to Arg 38 which is involved in Compound I formation (76).

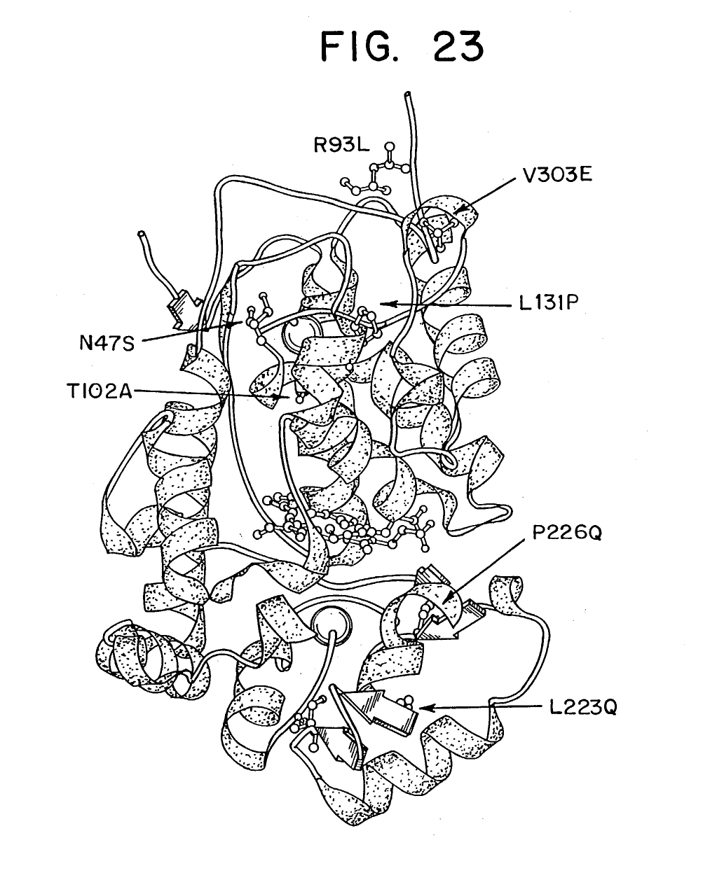

The mutations in HRP1-117G4 are preserved in the second and third generations. FIG. 23 shows the positions of amino acid substitutions in mutants HRP2-13A10 and HRP3-17E12. (Introduced mutations for HRP2-13A10 and HRP3-17E12 are shown. Mutations N47S and P226Q, possibly responsible for instability of HRP3-17E12 , are located near to the two Ca²⁺ ions, shown as black balls. Heme group is shown in the center of the enzyme.)

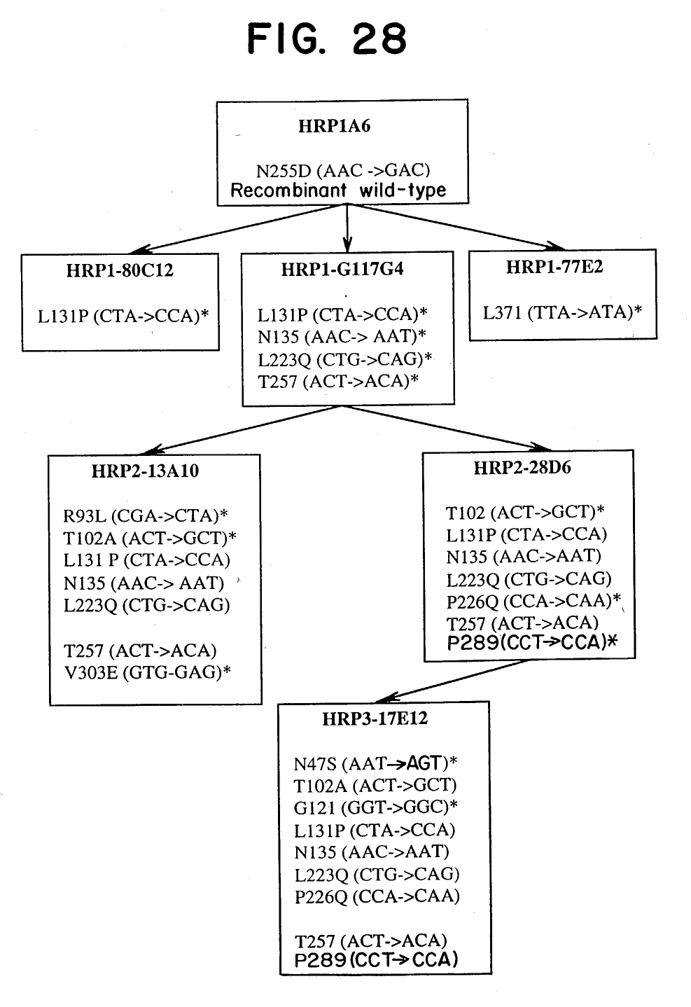

HRP2-13A10 contains three more mutations with respect to HRP1-117G4, all nonsynonymous: R93L, T102A, and V303E (FIG. 28). R93L is in the loop connecting helices C and D. T102A is part of helix D. It was the only buried mutation found, and is close to the active site. Finally, V303E is part of the long strand extending from helix J at the C-terminus of the protein. As illustrated in FIG. 23, HRP 2-13A10 has a concentration of mutations (R93L, L131P, V303E) on the surface of the enzyme. HRP2-28D6 contains two new nonsynonymous mutations with respect to HRP1-117G4: T102A and P226Q. P226Q is located in the same loop as L223Q. This mutant also contains a synonymous, or silent mutation; P289.

Third-generation HRP3-17E12 contains two more mutations with respect to the parent HRP2-28D6: N47S, and one synonymous mutation (FIG. 28). N47S is located in the loop that connects helix B and a 3₁₀-helix, and is solvent accessible. N47S lies between V46 and G48, which coordinate the distal Ca²⁺ ion, while P226Q is next to T225 and I228, which bind the proximal Ca²⁺ ion. Both Ca²⁺ ions are structurally coupled to the active site (47). Removal of calcium reduces the specific activity and dramatically reduces the thermal stability of the native enzyme (77). The relative instability of HRP3-17E12 with respect to its parent HRP2-28D6 may reflect disruption of calcium binding. HRP2-28D6, which also contains the P226Q mutation, also exhibits decreased thermal stability (FIG. 22).

The four synonymous mutations in HRP3-17E 12 can not be easily explained by changes in codon usage (Table I).

TABLE I
Codon Usage for S. cerevisiae.
[gbpln]: 11607 CDS's (5607643 codons)
fields: [triplet] [amino acid] [fraction] [frequency/thousand] ([number])

| | | | | | | | | | |
|---|---|---|---|---|---|---|---|---|---|
| UUU | F | 0.59 | 26.0 | (145919) | UCU | S | 0.27 | 23.6 | (132257) |
| UAU | Y | 0.56 | 18.8 | (105193) | UGU | C | 0.63 | 8.0 | (44640) |
| UUC | F | 0.41 | 18.2 | (102100) | UCC | S | 0.16 | 14.2 | (79697) |
| UAC | Y | 0.44 | 14.7 | (82257) | UGC | C | 0.37 | 4.7 | (26255) |
| UUA | L | 0.28 | 26.3 | (147731) | UCA | S | 0.21 | 18.8 | (105246) |
| UAA | * | 0.48 | 1.0 | (5515) | UGA | * | 0.30 | 0.6 | (3449) |
| UUG | L | 0.29 | 27.1 | (152214) | UCG | S | 0.10 | 8.6 | (48065) |
| UAG | * | 0.23 | 0.5 | (2622) | UGG | W | 1.00 | 10.3 | (57933) |
| CUU | L | 0.13 | 12.2 | (68291) | CCU | P | 0.31 | 13.6 | (76179) |
| CAU | H | 0.64 | 13.7 | (77033) | CGU | R | 0.15 | 6.5 | (36410) |
| CUC | L | 0.06 | 5.4 | (30110) | CCC | P | 0.15 | 6.8 | (38132) |
| CAC | H | 0.36 | 7.8 | (43767) | CGC | R | 0.06 | 2.6 | (14527) |
| CUA | L | 0.14 | 13.4 | (75212) | CCA | P | 0.41 | 18.2 | (102040) |
| CAA | Q | 0.69 | 27.5 | (154138) | CGA | R | 0.07 | 3.0 | (16913) |
| CUG | L | 0.11 | 10.4 | (58408) | CCG | P | 0.12 | 5.3 | (29668) |
| CAG | Q | 0.31 | 12.2 | (68271) | CGG | R | 0.04 | 1.7 | (9781) |
| AUU | I | 0.46 | 30.2 | (169286) | ACU | T | 0.35 | 20.2 | (113345) |
| AAU | N | 0.59 | 36.0 | (201802) | AGU | S | 0.16 | 14.2 | (79390) |
| AUC | I | 0.26 | 17.1 | (95862) | ACC | T | 0.22 | 12.6 | (70604) |
| AAC | N | 0.41 | 24.9 | (139844) | AGC | S | 0.11 | 9.7 | (54161) |
| AUA | I | 0.27 | 17.8 | (99708) | ACA | T | 0.30 | 17.7 | (99504) |
| AAA | K | 0.58 | 42.1 | (236119) | AGA | R | 0.48 | 21.3 | (119340) |
| AUG | M | 1.00 | 20.9 | (117085) | ACG | T | 0.14 | 8.0 | (44674) |
| AAG | K | 0.42 | 30.8 | (172691) | AGG | R | 0.21 | 9.3 | (51898) |
| GUU | V | 0.39 | 22.0 | (123387) | GCU | A | 0.38 | 21.1 | (118280) |
| GAU | D | 0.65 | 37.8 | (212077) | GGU | G | 0.47 | 23.9 | (134191) |
| GUC | V | 0.21 | 11.6 | (65005) | GCC | A | 0.22 | 12.6 | (70625) |
| GAC | D | 0.35 | 20.4 | (114141) | GGC | G | 0.19 | 9.7 | (54466) |
| GUA | V | 0.21 | 11.7 | (65852) | GCA | A | 0.29 | 16.2 | (90764) |
| GAA | E | 0.71 | 45.9 | (257286) | GGA | G | 0.22 | 10.9 | (61244) |
| GUG | V | 0.19 | 10.7 | (59828) | GCG | A | 0.11 | 6.1 | (34443) |
| GAG | E | 0.29 | 19.1 | (107249) | GGG | G | 0.12 | 6.0 | (33519) |

Coding GC 39.72%
1st letter GC 44.60%
2nd letter GC 36.59%
3rd letter GC 37.98%.
Genetic code 1: Standard.

Three mutations, AAC→AAT, ACT→ACA, and CCT→CCA, resulted in little changes in the frequencies of codons used, while for GGT→GGC, a more frequently used codon (GGT, 23.9 per thousand) was replaced by a less frequent one (GGC, 9.7 per thousand). Nor do codon frequencies for the nonsynonymous mutations reveal strong differences in codon usage. The only exception is mutation R93L, where a less frequent codon (CGA, 3 per thousand) is replaced by a more frequent one (CTA, 13.3 per thousand).

### EXAMPLE 5

### Comparison of HRP Mutant Activity

The total and residual HRP activity of recombinant wild-type HRP1A6, and mutants HRP1-117G4, HRP1-77E12, HRP1-80C12, HRP2-13A10, HRP2-28D6, and HRP3-17E12 were compared with wild type. Cells were grown in 5 ml cultures at 30° C. for 64 hours. Activities were measured on ABTS as described in above (39). Residual activity (A_{resid}) was measured after 10 min incubation at 50° C. Thermostability was determined from the ratio of residual to initial activity (FIG. 22). HRP2-28D6 and HRP3-17E12 both show a substantial loss of activity after incubation, whereas wild type and the other mutants retained 75-98% activity.

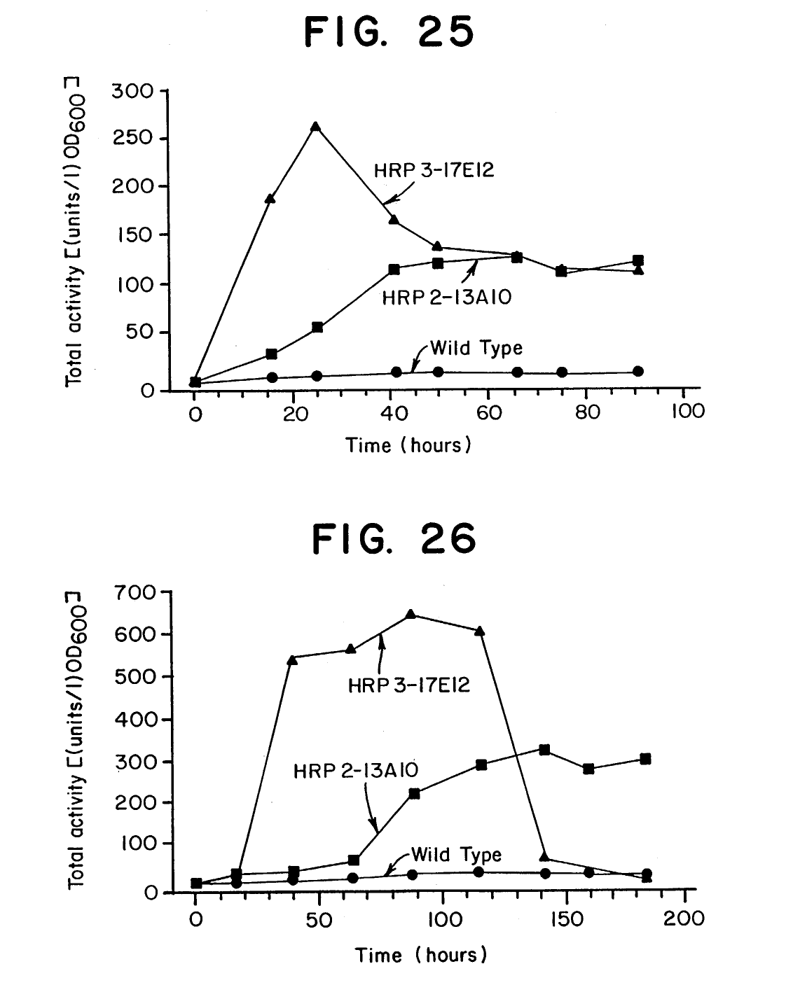

Expression studies with wild type and the two mutants in YEPD media showed that the amount of HRP activity in S. cerevisiae depends on the time of growth (FIG. 25). 100 ml cultures were inoculated with 24-hour cultures grown in selective media. After 25 hours of growth, the total activity for HRP3-17E12 was 40-fold greater than wild type and 4.8-fold over HRP 2-13A10. After this time point there was no significant further increase of activity for this mutant, whereas activity for wild type and HRP2-13A10 continued to increase. OD₆₀₀ values of the cultures did not differ significantly, and the increase in total activity cannot be attributed to changes in growth rates.

In these Examples, three rounds of directed evolution by random point mutagenesis and screening produced a 40-fold increase in total HRP activity in the S. cerevisiae culture supernatant compared to wild type, as measured on ABTS (2,2′-azino-bis(3-ethylbenzthiazoline-6-sulfonic acid) (260 units/l/OD₆₀₀). Genes from wild type and two high-activity clones were expressed in P. pastoris, where the total ABTS activity reached 600 units/l/OD₆₀₀ in shake flasks. The mutants show up to 5.4-fold higher specific activity towards ABTS and 2.3-fold higher towards guaiacol.

### EXAMPLE 6

### HRP Expression in Pichia pastoris

A. Pichia Cloning Vector

Genes encoding HRP-C (wild type), HRP2-13A10 and HRP3-17E12 were cloned into the Pichia secretion vector pPICZ_B. In this construct, pPICZ_B-HRP (FIG. 24), HRP is fused to the α-factor signal peptide, and expression is induced with methanol. The HRP genes were cloned into pPICZ_B containing the α-factor secretion signal and the methanol-inducible P_{AOX1} promoter. Plasmid pPICZ_B was restricted with Pst I, blunt-ended with T4 DNA polymerase, further digested with EcoR I and purified. The coding sequences for the HRP variants were obtained from the corresponding pYEXS1-HRP plasmids by PCR techniques using the proofreading polymerase Pfu and following two primers:

| | |
|---|---|
| 5′-TCAGTTAACCCCTACATTC-3′ | [SEQ ID NO:27] |
| (forward) and | |
| 5′ CCACCACCAGTAGAGACATGG-3′ | [SEQ ID NO:28] |
| (reverse). | |

The PCR products were restricted with EcoR I and ligated into pPICZ_B, yielding pPICZ_B-HRP (FIG. 24), in which the HRP genes were placed immediately downstream of the α-factor signal. The ligation products were transformed into E. coli strain XL10-Gold and selected on low salt LB medium (1% tryptophan, 0.5% yeast extract, 0.5% NaCl, pH adjusted to 7.5) supplemented with 25 1g/ml Zeocin. Colonies were screened for the HRP genes by colony PCR reactions (53) with two primers:

| | |
|---|---|
| 5′-GAGAAAAGAGAGGCTGAAGCTC-3′ | [SEQ ID NO:29] |
| (forward) and | |
| 5′-TCCTTACCTTCCAATAATTC-3 | [SEQ ID NO:30] |
| (reverse). | |

The forward primer contained the last three nucleotides of the signal sequence and the first nucleotide of the HRP sequence (underlined), which ensured that the positive colonies carried the full-length HRP genes in the correct orientation. Plasmids were isolated and transformed into Pichia by electroporation according to the supplier's instructions (Invitrogen). Before transformation, plasmids were linearized with Pme I. The linearized vectors were integrated into the Pichia genome upon transformation via homologous recombination. The transformed cells were plated on YPDS medium (1% yeast extract, 2% peptone, 2% glucose, 1M sorbitol) supplemented with 100 μg/ml Zeocin. For each HRP construct, typically 4-6 transformants were picked and purified on new YPDS plates (supplemented with 100 μg/ml Zeocin) to isolate single colonies, which were then screened to identify the clones with the highest levels of HRP activity. Pichia strain X-33 was used in all experiments. It was determined in initial tests that X-33 (Mut⁺) afforded significantly better HRP expression than KM17 (Mut^{S}). Activity levels in X-33 were up to threefold higher than those for KM 17.

B. HRP Expression in P. pastoris.

Pichia cells were grown at 30° C. in shake flasks. pPIZ_B-HRP-harboring cells were first grown overnight in BMGY (1% yeast extract, 2% peptone, 100 mM potassium phosphate, pH 6.0, 1.34% YNB, 4×10-5% biotin, 1% glycerol) supplemented with 1% casamino acids to an OD600 of 1.2-1.6. The cells were then pelleted and resuspended to an OD600 of 1.0 in BMMY medium (identical to BMGY except 0.5% methanol in lieu of 1% glycerol) supplemented with 1% casamino acids. Growth was continued for another 72-150 h. Sterile methanol was added every 24 h to maintain induction conditions. HRP levels in the supernatants peaked around 80-90 h post-induction (at which time the OD600 reached about 8.0-10.0). Where applicable, at the point of induction, 1.0 mM vitamin B1, 1.0 mM δ-ALA, and 0.5 ml/l trace element mix (0.5 g/l MgCl2, 30 g/l FeCl2.6H2O, 1 μg/l ZnCl2.4H2O, 0.2 g/l CoCl2.6H2O, 1 g/l Na2MoO4.2H2O, 0.5g/l CaCl2.2H2O, 1 g/l CuCl2, and 0.2 g/l H2BO3) were added to the growth medium.

The total activity in the Pichia culture supernatant increased ˜5-6 fold compared to S. cerevisiae for all three HRP variants. When normalized by OD600 to account for differences in cell density, this increase is about 3-fold. Typical activity profiles for the P. pasloris cultures are shown in FIG. 26. The total activity for HRP3-17E12 decreased rapidly after 150 hours, presumably due to instability of this mutant and/or its increased susceptibility to degradation by proteases in the culture broth, since the P. pastoris strain used is not protease-deficient.

Addition of trace metal elements, the heme synthesis intermediate δ-aminolevulinic acid and vitamin supplements such as thiamine are known to improve the yields of holoenzymes of heme-containing proteins in E. coli. (67-71). Their addition to the Pichia growth medium led to a 32% increase in HRP3-17E12 activity detected in the supernatant at peak levels (80-90 hr post-induction.

C. Purification of HRP Wild Type, HRP2-13A10 and HRP3-17E12 from P. pastoris.

Yeast cultures were harvested at the time point corresponding to the highest activity for HRP3-17E12 and centrifuged at 5000 rpm on a GS-3 rotor for 20 min. 30% (NH4)2SO4 was added to the supernatant, which was loaded on a Phenylsepharose 6 Fast Flow column (Pharmacia) equilibrated with 30% (NH4)2SO4 in 50 mM phosphate buffer pH 7.0, flow rate 1.5ml/min. Protein was eluted in a 30% to 0% (NH4)2SO4 gradient. Fractions with the highest ABTS activity were combined and concentrated to 1-3.2 ml with an Amicon membrane filtration device (Millipore Corp., Bedford, Mass.). The protein sample was then loaded on a Sephacryl 200 (Pharmacia) column equilibrated with 50 mM phosphate buffer pH 7.0 and 50 mM KCl and eluted in the same buffer. Flow rate was 0.25 ml/min. Fractions with the highest peroxidase activity were pooled, dialyzed against 50 mM phosphate buffer pH 7.0 and applied to a MonoQ (Pharmacia) column and eluted in a 0-1 M NaCl gradient in 50 mM phosphate buffer (pH 7.0) to remove impurities.

Total protein concentration was determined after Bradford (72) with BSA as standard. The RZ values (A404/A280) as measurement for the purity (73) were between 1.2-1.9 for the final protein samples.

Protein samples collected after the final purification step showed an RZ (A404/280) value between 1.2-1.9. The RZ value measures heme content using the aromatic amino acid content as reference and is a measure of the purity of HRP preparations (73). SDS gels did not show a discrete band but rather a smear in the range 66-100 kDa, indicating a protein with high mannose-type oligosaccharides of varying grades of polymerization, as observed previously for a staphylokinase expressed in P. pastoris (74).

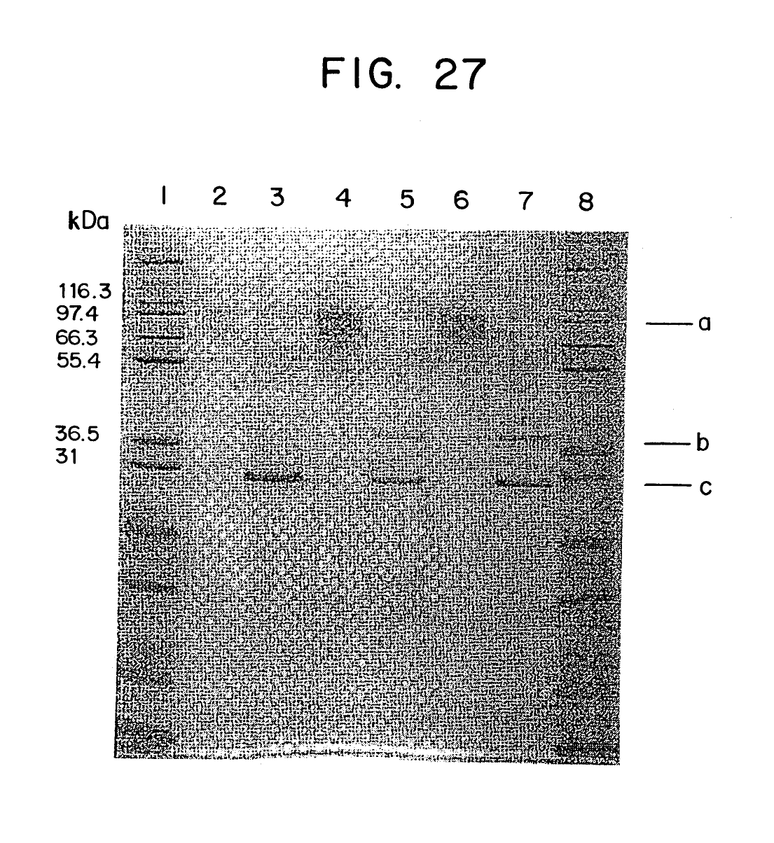

Deglycosylation with EndH caused the smear to collapse to a single discrete band at about 37,000 Da (FIG. 27). Samples of combined MonoQ fractions before and after deglycosylation with EndH were subjected to SDS/PAGE under reducing conditions. Arrows show (a) glycosylated HRP, (b) deglycosylated HRP, (c) deglycosylating enzyme EndH. Samples were: (1) MW standard; (2) HRP-C wild type (0.15 μg ); (3) HRP-C wild type deglycosylated; (4) HRP2-13A10 (0.3 μg); (5) HRP2-13A10 deglycosylated; (6) HRP3-17E12 (0.3 μg); (7) HRP3-17E12 deglycosylated; (8) MW standard. EndH leaves one GlcNAc residue per glycosylation site. This accounts for the difference in size between the non-glycosylated enzyme (34,520 Da) and the EndH-deglycosylated enzyme (about 37,000 Da).

Specific activities of the purified enzymes towards ABTS and guaiacol are summarized in Table II. The specific activities of HRP-C (Type VI, Sigma), recombinant wild type HRP-C, HRP2-13A10 and HRP3-17E12 expressed in P. pastoris and purified from the supernatant. ABTS activity was determined using 0.5 mM ABTS and 2.9 mM H2O2, pH 4.5, and guaiacol activity using 5 mM guaiacol and 1 mM H2O2, pH 7.0. Protein concentration was determined after Bradford (72) with bovine serum albumin as standard. Reported are the means of three separate experiments; standard deviations are 8-10%.

TABLE II

| Enzyme | A₄₀₄/A₂₈₀ | ABTS (U/mg) | Guaiacol (U/mg) |
|---|---|---|---|
| HRP Type VI (Sigma) | 3.5 | 1292 | 205 |
| HRP-C WT | 1.3 | 377 | 82 |
| HRP2-13A10 | 1.23 | 2053 | 191 |
| HRP3-17E12 | 1.9 | 1049 | 96 |

Specific activities on both substrates are higher for the two mutants compared to the recombinant wild-type HRP. The increase is about 5.4-fold for HRP2-13A10 and 2.8-fold for HRP3-17E12 for ABTS. Activity increases are less toward guaiacol, indicating a slight change in substrate specificity. HRP3-17E12 loses 50-60% of its activity after two hours at room temperature, and has lost its activity completely after incubation at 50° C. for 10 min while HRP2-13A10, still retains 85% activity under these conditions. This is consistent with the stabilities of the mutants expressed in S. cerevisiae (FIG. 22). From the total activities in the Pichia cultures and the specific activities of the purified HRP's, total expression levels can be estimated: ˜0.6 mg of HRP/1 for wild type, 0.9 mg/l for HRP3-12A10 and 5.2 mg/l for HRP2-17E12.

The specific activities of the recombinant HRPs are lower than the most highly purified commercial HRP preparation (Sigma Type VI), which contains ca. 75% HRP-C isozyme. The RZ values of the recombinant enzyme preparations (1.2-1.9) are lower than for the native HRP-C (3.5), indicating contamination with inactive heme-free enzyme (26). It is also possible that the strong glycosylation (48-65 % judged from SDS gels) of the recombinant HRP decreases its specific activity (75).

It will be appreciated that useful mutants can be rapidly obtained by successive rounds of error-prone PCR in the directed evolution methods described herein, and further, different mutations can be expected in different generations or evolutionary cycles starting with the same parent. In the Examples, a large fraction of clones in the first generation HRP random mutant library were prepared using error-prone PCR at low error rate (100 mM manganese). The resulting mutants showed activities similar to wild type, indicating that a significant fraction of the single mutations are neutral with respect to total expressed enzyme activity. For the HRP mutants herein, evolution is more efficient using higher error rates. For example, a preferred mutation rate is the rate associated with 0.35 mM concentration of manganese in the PCR reaction, which generates a library of mutant genes with predominately 4-6 base mutations per gene (although there will be genes with fewer than 4 and more than 6). In theory, the higher error rate can be tolerated because many of the mutations are effectively silent, and mutations that improve the target property are not lost in a background of deleterious mutations. Thus random mutagenesis in the second and third generations is preferably carried out under conditions expected to yield 4-6 base mutations per 1 Kb. The higher error-rate was also coupled with a pre-screen on membranes, which facilitated identification of the most active clones among a larger number mutants (about 100,000).

A further potential benefit to utilizing a slightly higher mutation rate is that the neutral mutations can become useful in subsequent generations by either providing a bridge for generating new types of amino acid substitutions not accessible by single-base substitution or by synergy with newly-created mutations. Here, the more active mutants identified in generations 2 and 3 contain between two and four base substitutions (FIG. 28).

None of the mutations found to increase the total enzyme activity in S. cerevisiae were previously described as essential for HRP activity: key catalytic residues are Arg 38, Phe 41, His 42, Asn 70, His 170, and Asp 247, while Phe 68, Phe 142 and Phe 179 have been found to be important for binding of aromatic substrates (78). Most of the present mutations found to contribute to increased secreted HRP activity were located in loop regions and on the surface of the protein, a common theme of mutations identified in directed evolution studies (79-81). Two mutations adjacent to the Ca²⁺ binding sites may be responsible for the accompanying destabilization of the enzyme in the 2^{nd} and 3^{rd} generations.

Comparing the total activities of the HRP's in the S. cerevisiae supernatant, a 4.8-fold increase was obtained for HRP2-13A10, and 40-fold for HRP3-17E12, with respect to the wild type. The mutants that were better expressed in S. cerevisiae also generated higher total HRP activity in the P. pastoris cultures. A similar observation of a correlation between increased total activity for mutants expressed in S. cerevisiae and increased total and specific activity of enzyme expressed in a different host (Aspergillus niger) was recently described for another (fungal) peroxidase (82).

Purification and characterization of the HRPs produced in P. pastoris revealed that their specific activities were higher than (recombinant) wild type's (Table I). These results suggest that the improvement in the total activities obtained for HRP mutants in S. cerevisiae may also be, at least in part, due to higher specific activities. However, the enzymes made in the two organisms will differ in their glycosylation patterns. P. pastoris adds shorter N-linked high-mannose saccharides to proteins than S. cerevisiae, which hyperglycosylates (83). Therefore, the specific activities of a given mutant may differ, depending on whether it is produced in P. pastoria or in S. cerevisiae. Due to the lower total expressed activity, mutant enzymes produced in S. cerevisiae have not been adequately purified in order to identify what fraction of the increased total activity is due to changes in expression levels versus specific activity.

The mutations discovered by directed evolution may also influence the folding and secretion of the enzyme in yeast. Protein secretion is directed by a signal sequence, which mediates the translocation of the peptide into the endoplasmatic reticulum (ER) and is subsequently removed. Within the ER, the protein is folded and glycosylated. Glycosylation in the ER influences protein folding, oligomerization and quality control (84-86). Correct folding of the secreted protein is required for export competence. Misfolding can result in retention in the ER and degradation, and can dramatically decrease the secretion level (83). The next step in the secretion process is the transport of correct folded proteins from the ER to the Golgi apparatus, where modifications to the glycosyl structures take place. From the Golgi, proteins are packed into secretory vesicles and are delivered to the cell surface. It is possible that the mutations influence these different steps of the secretion process to improve the release of functional protein into the supernatant.

HRP2-28D6 and HRP3-17E12 are less stable compared with wild-type HRP (FIG. 22). It is to be expected that further rounds of evolution will stabilize the enzyme and improve the total secreted activity e.g. by including stability in the screens (80, 87, 88).

### BIBLIOGRAPHY

1. Ausubel, F. M., Brent, R., Kingston, R. E., Moore, D. D., Seidman, J. S., Smith, J. A. & Struhl, K. , Current Protocols in Molecular Biology,. Greene Publishing Associates And Wiley-Interscience, New York, (1987).

2. Bagger, S. & Williams, R. J. P., Acta Chem. Scand., 25, 976-987, (1971).

3. Burke, J. F., Smith, A., Santama, N., Bray, R. C., Thorneley, R. N., Dacey S., Griffiths, J., Catlin, G. & Edwards, M., (1989).

4. Chriswell, D. J., European Patent 029968-Al., (1988).

5. Cregg, J. M., Vedvick, T. S. & Raschke, W. C., Bio/Technology, 11, 905-910., (1993).

6. Dordick, J. S., Marletta, M. A. & Klibanov, A. M. Biotechnol. Bioeng., 30, 31-36., (1987).

7. Gray, J. S. S., Yang, B. Y. & Montgomery, R., Carbohyd. Res., 311, 61-69., (1998).

8. Hartmann, C. & Ortiz de Montallano, P. R., Arch. Biochem. Biophys., 15, 61-72., (1992).

9. Holland, H. L., Organic synthesis vith oxidative enzymes. VCH publishers, New York. pp. 341-383., (1992).

10. Jayaraman, K., Fingar, S. A., Shah, J. & Fyles, J., Proc. Nati. Acad. Sci. USA, 88, 4084-4080.

11. Joo, H., Chae, H. J., Yeo, J. S., & Yoo, Y. J., Proc. Biochem., 32, 291-296., (1997).

12. Joo, H., Y. J. Yoo, & Dordick, J. S., Korean J. Chem. Eng., 15, 362-374., (1998).

13. Kedderis, G. L., & Hollenberg, P. F., J. Biol. Chem., 258, 8129-8138., (1983).

14. Cleland, J. L; Wang, D. I. C. Bio/Technology 8, 1274 (1990).

15. Bernarderz-Clark, E. D.; Georgiou, G. Inclusion Bodies and Recovery of Proteins from the Aggregated States. In Protein Refolding; Bernarderz-Clark, E. D., Georgiou, G., Eds,; ACS: Washington, D. C. p. 1-20 (1990).

16. Thatcher, D. R.; Hitchcock, A. Protein Folding in Biotechnology. In Mechanisms of Protein Folding; Pain, R. H., Ed.; IRL Press: Oxford p. 229-261 (1994).

17. Parekh, R.; Forrester, K.; Wittrup, D. Protein Expres. Purif. 6, 537 (1995).

18. Arnold, F. H. Accounts Chem. Res. 31, 125 (1998).

19. Mitraki, A.; King, J. FEBS Lett. 307, 20 (1992).

20. Zhang, J. X.; Goldenberg, D. P. Biochemistry 32, 14075 (1993).

21. Wetzel, R.; Perry, L. P.; Veilleux, C. Bio/Technology 9, 731 (1991).

22. Knappik, A.; Pluckthun, A. Protein Eng. 8, 81 (1995).

23. Crameri, A.; Whiteborn, E. A.; Tate, E.; Stemnmer, W. P. C. Nature Biotechnol. 14, 315 (1996).

24. Tams, J. W.; Welinder, K. G. FEBS Lett. 421, 234 (1998).

25. Ortlepp, S. A.; Pollard-Knight, D.; Chiswell, D. J. J. Biotechnol. 11, 353 (1989).

26. Smith, A. T. et al. J. Biol. Chlem. 265, 13335-13343 (1990).

27. Egorov, A. M.; Gazaryan, I. G.; Savelyev, S. V.; Fechina, V. A.; Veryovkin, A. N.; Kim, B. B. Ann. N.Y. Acad. Sci. 646, 35 (1991).

28. Moore, J. C.; Arnold, F. H. Nature Biotechnol. 14, 458 (1996).

29. Fitzgerald, M. M.; Churchill, M. J.; McRee, D. E.; Goodin, D. B. Biochemistry 33, 3807 (1994).

30. Goodin, D. B.; Davidson, M. G.; Roe, J. A.; Mauk, A. G.; Smith, M. Biochemistry 30, 4953 (1991).

31. De Sutter, K.; Hostens, K.; Vandekerckhove, J.; Fiers, W. GENE 141, 163 (1994).

32. Sambrook, J.; Fritsch, E. F.; Maniatis, T. Molecular Cloning: A Laboratory Manual; Cold Spring Harbor Laboratory: New York (1989).

33. Caldwell, R. C.; Joyce, G. F. PCR Methods Applic. 2, 28 (1992).

34. Beckman, R. A.; Mildvan, A. S.; Loeb, L. A. Biochemistry 24, 5810 (1994).

35. Shafikhani, S.; Siegel, R. A.; Ferrari, E.; Schellenberger, V. Biotechniques 23, 304 (1997).

36. Stemmer, W. P. C. Proc. Natl. Acad. Sci. USA 91, 10747 (1994).

37. Zhao, H. M.; Arnold, F. H. Nucleic Acids Res. 25, 1307 (1997).

38. Carbon, J.; Clarke, L.; Ilgen, C.; Ratzkin, B. The Construction and Use of Hybrid Plasmid Gene Banks in Escherichia coli. In Recombinant Molecules: Impact on Science and Society; Beers, R. F. J., Bassett, E. G., Eds; Raven Press: New York, pp 355-378 (1977).

39. Shindler, J. S.; Childs, R. E.; Bardsley, W. G. Eur. J. Biochem. 65, 325 (1976).

40. Lei, S. P.; Lin, H. C.; Wang, S. S.; Callaway, J.; Wilcox, G. J. Bacteriol. 169, 4379 (1987).

41. Better, M.; Chang, C. P.; Robinson, R. R.; Horwitz, A. H. Science 240, 1041 (1988). 42. Goshorn, S. C.; Svensson, H. R.; Kerr, D. E.; Somerville, J. E.; Senter, P. D.; Fell, H. P. Cancer Res. 53, 2123 (1993).

43. Rathore, D.; Nayak, S. K.: Batra, J. K. FEBS Lett. 392, 259 (1996).

44. Studier, F. W.; Rosenberg, A. H.; Dunn, J. J.; Dubendorff, J. W. Meth. Enzymol. 185, 60 (1990).

45. Ostermeier, M.; Desutter, K.; Georgiou, G. Eukaryotic J. Biol. Chern. 271, 10616 (1996).

46. Savenkova, M. I.; Kuo, J. M.; Ortiz de Montellano, P. R. Biochemistry 37, 10828 (1998).

47. Gajhede, M.; Schuller, D. J.; Henriksen, A.; Smith, A. T.; Poulos, T. L. Nature Struct. Biol. 4, 1032 (1997).

48. Anfinsen, C. B. Science 181, 223 (1973).

49. Schein, C. H. Bio/Technology 8, 308 (1990).

50. Martineau, P.; Jones, P.; Winter, G. J. Mol. Biol. 20, 117 (1998).

51. Stemmer, W. P. C. et al., Biotechniques 14, 256 (1992).

52. Arkin, A. and Youvan, D. C. Proc. Natl. Acad. Sci. USA 89, 7811 (1992).

53. Oliphant, A. R. et al., Gene 44, 177 (1986).

54. Hermes, J. D. et al., Proc. Natl. Acad. Sci. USA 87, 696 (1990).

55. Delagrave et al. Protein Engineering 6, 327 (1993).

56. Delagrave et al. Bio/Technology 11, 1548 (1993).

57. Goldman, E. R. and Youvan D. C. Bio/Technology 10,1557 (1992).

58. Leung, D. W. et al., Technique 1, (1989).

59. Gramm, H. et al., Proc. Natl. Acad. Sci. USA 89, 3576 (1992).

60. Castelli, M. C. et al., Gene 142, 113 (1994).

61. Gietz, D., Schiestl, R. H., Willems, A., Woods, R. A., Yeast 11, 355 (1995).

62. Welinder, K. G., Eur. J. Biochem 96, 483-502 (1979).

63. Sirotkin, K. J. Theor. Biol. 123, 261 (1986).

64. Glover, D. M. (ed.), DNA Cloning: A Practical Approach, MRL Press, Ltd., Oxford, U.K. Vol. I, II., (1985).

65. Schatz, P. J. Et al., Annu. Rev. Genet. 24, 215-248 (1990).

66. Gussow, D. & Clackson, T. (1989) Nucleic Acids Res. , 17, 4000-4000.

67. Gillam, E. M., Guo, Z., Martin, M. V., Jenkins, C. M. & Guengerich, F. P. (1995) Arch. Biochem. Biophys., 319, 540-550.

68. Guengerich, F. P., Martin, M. V., Guo, Z. & Chun, Y. J. (1996) Meth. Enzymol., 272,35-44.

69. Joo, H., Lin, Z. & Arnold, F. H. (1999) Nature , 399, 670-673.

70. Joo, H., Arisawa, A., Lin, Z. & Arnold, F. H. (1999) Chem. Biol., 6, 669-706.

71. Khosla, C., Curtis, J E, Demodena, J., Rinas, U. & Bailey, J E, Bio/Technology, 8, 849- 853, (1990).

72. Bradford, M., Anal. Biochem., 72, 248-254., (1978).

73. Dunford, H. B., Peroxidases in Chemistry and Biology, Vol 2. pp. 1-24., (1991).

74. Miele, R. G., Prorok, M., Costa V. A. & Castellino F. J. , J. Biol. Chem., 274, 7769-7776., (1999).

75. Gazaryan, I. G., LABPV Newsletters , 4, 8-15., (1994).

76. Rodriguez-Lopez, J. N., Smith, A. T., and Thorneley, R. N. F., J. Biol. Chem., 271, 4023-4030., (1995).

77. Haschke, R. H. & Friedhoff, J. M., Biochim. Biophys. Res. Commun., 90, 1039-1042., (1978).

78. Smith, A. T., & Veitch, N. C., Curr. Opin. Chem. Biol. , 2, 269-278., (1998).

79. Chen, K. & Arnold, F. H., Proc. Natl. Acad. Sci. USA , 90, 5618-5622., (1993).

80. Giver, L., Gershenson, A., Freskgard, P -O. & Arnold, F. H., Proc. Natl. Acad. Sci. USA 95, 12809-12813., (1998).

81. Yano, T., Oue, S. & Kagamiyama, H., Proc. Natl. Acad. Sci. USA , 95, 5511-5515., (1998).

82. Cherry, J. R., Lamsa, M. H., Schneider, P., Vind, J., Svendson, A., Jones, A., & Pederson, A. H., Nat. Biotechnol. , 17, 379-384., (1999).

83. Romanos, M. A., Scorer, C. A. & Clare J. J., Yeast , 8, 423-488., (1992).

84. Fiedler, K. & Simons, K., Cell , 81, 309-312., (1995).

85. Nagayama, Y., Namba, H., Yokoyama, N., Yamashita, S. & Niwa, M., J. Biol. Chem., 273, 33423-33428., (1998).

86. Helenius, A., Mol. Biol. Cell. , 5, 253-265., (1994).

87. Miyazaki, K., Wintrode, P. L., Grayling, R. A., Rubingh, D. N. & Arnold, F. H., J. Mol. Biol. in press., (2000).

88. Zhao, H. M. & Arnold, F. H., Protein Eng. 12, 47-53., (1999).

89. U.S. Pat. No. 5,605,793.

90. U.S. Pat. No. 5,741,691.

91. U.S. Pat. No. 5,811,238.

92. U.S. Pat. No. 5,830,721

93. WO 98/42832.

94. WO 95/22625.

95. WO 97/20078

96. WO 95/41653.

97. WO 98/27230.

We claim:

1. A method of obtaining and improving the production of a functional polypeptide by a host cell comprising the steps of:
(a) providing at least one parent polynucleotide encoding a parent polypeptide,
(b) altering the nucleotide sequence of the parent polynucleotide to produce a population of mutant polynucleotides;
(c) transforming host cells with the mutant polynucleotides to express polypeptides; and
(d) screening the polypeptides for a functional polypeptide having at least one modified property.

2. The method of claim 1 wherein the parent polypeptide is characterized by at least one of the following:
(a) contains a disulfide bridge structure;
(b) contains a heme group; or
(c) is associated with a heme group.

3. The method of claim 1 wherein the parent polypeptide is characterized by at least one of the following:
(a) is expression-resistant
(b) forms inclusion bodies when expressed in the host cells;
(c) is produced in a non-functional form when over-expressed in the host cells and is produced in a functional form when under-expressed in the host cells; or
(d) is over-expressed under the control of an inducible promoter in the presence of an inducer, and is under-expressed under the control of an inducible promoter in the absence of an inducer.

4. The method of claim 1, wherein the parent polypeptide is a peroxidase enzyme.

5. The method of claim 4, wherein the peroxidase enzyme is selected from a horseradish peroxidase and a cytochrome c peroxidase.

6. The method of claim 1, wherein the alteration of the parent polynucleotide sequence is performed by at least one of the following:
(a) random mutagenesis;
(b) site-specific mutagenesis; or
(c) DNA shuffling.

7. The method of claim 6, wherein the random mutagenesis comprises error-prone polymerase chain reaction (PCR).

8. The method of claim 7, wherein the error-prone polymerase chain reaction employs unbalanced nucleotide concentrations.

9. The method of claim 7, wherein the error-prone polymerase chain reaction employs manganese ions in a concentration of about 0.15 to about 0.35 mM.

10. The method of claim 7, wherein the error-prone polymerase chain reaction employs manganese ions in a concentration of up to about 0.15 μM.

11. The method of claim 7, wherein the polymerase chain reaction generates an error rate of up to 2 mutations per polynucleotide.

12. A method of claim 7, wherein the polymerase chain reaction generates an error rate of up to 6 mutations per polynucleotide.

13. The method of claim 1 wherein the host cells are facile host cells.

14. A method of claim 13 wherein the facile host cells are selected from yeast and bacteria.

15. The method of claim 14 wherein the host cells are E. coli cells.

16. The method of claim 14 wherein the host cells are selected from S. cerevisiae cells and P. pastoris cells.

17. The method of claim 1, wherein the host cells are transformed by vectors comprising a mutant polynucleotide and a signal sequence that directs the secretion of polypeptides encoded by the mutant polynucleotide.

18. The method of claim 17, wherein the signal sequence is the PelB signal sequence.

19. The method of claim 1, wherein the screening step comprises screening for at least one of the following properties:
(a) the biological activity of the polypeptide;
(b) the stability of the polypeptide; or
(c) the yield of expressed polypeptide
(d) the yield of expressed functional polypeptide.

20. The method of claim 1, wherein screening comprises pre-screening for mutant colonies using nitrocellulose membranes.

21. A functional polypeptide produced according to the method of claim 1.

22. A method of obtaining and improving the production of a functional polypeptide by a host cell comprising the steps of:
(a) providing an original parent polynucleotide encoding a parent polypeptide;
(b) altering the nucleotide sequence of the parent polynucleotide to produce a population of mutant polynucleotides;
(c) transforming host cells with the mutant polynucleotides to express polypeptides;
(d) screening the polypeptides for a functional polypeptide having at least one modified property;
(e) repeating steps (b)-(d) at least once to produce an additional generation of polypeptides, wherein a mutant polynucleotide encoding a functional polypeptide is employed as a new parent polynucleotide.

23. The method of claim 22 wherein the original parent polypeptide is characterized by at least one of the following:
(a) contains a disulfide bridge structure;
(b) contains a heme group; or
(c) is associated with a heme group.

24. The method of claim 22 wherein the original parent polypeptide is characterized by at least one of the following:
(a) is expression-resistant
(b) forms inclusion bodies when expressed in the host cells;
(c) is produced in a non-functional form when over-expressed in the host cells and is produced in a functional form when under-expressed in the host cells; or
(d) is over-expressed under the control of an inducible promoter in the presence of an inducer, and is under-expressed under the control of an inducible promoter in the absence of an inducer.

25. The method of claim 22, wherein the original parent polypeptide is a peroxidase enzyme.

26. The method of claim 25, wherein the peroxidase enzyme is selected from a horseradish peroxidase and a cytochrome c peroxidase.

27. The method of claim 22, wherein the alteration of the parent polynucleotide sequence in at least one cycle is performed by at least one of the following:
(a) random mutagenesis;
(b) site-specific mutagenesis; or
(c) DNA shuffling.

28. The method of claim 27, wherein the random mutagenesis in at least one cycle comprises error-prone polymerase chain reaction (PCR).

29. The method of claim 27, wherein the error-prone polymerase chain reaction in at least one cycle employs unbalanced nucleotide concentrations.

30. The method of claim 27, wherein the error-prone polymerase chain reaction in least one cycle employs manganese ions in a concentration of about 0.15 to about 0.35 mM.

31. The method of claim 27, wherein the error-prone polymerase chain reaction in least one cycle employs manganese ions in a concentration of up to about 0.15 mM.

32. The method of claim 27, wherein the polymerase chain reaction in least one cycle generates an error rate of up to 2 mutations per polynucleotide.

33. A method of claim 27, wherein the polymerase chain reaction in least one cycle generates an error rate of up to 6 mutations per polynucleotide.

34. The method of claim 22 wherein the host cells in least one cycle are facile host cells.

35. A method of claim 34 wherein the facile host cells in least one cycle are selected from yeast and bacteria.

36. The method of claim 35 wherein the host cells in least one cycle are E. coli cells.

37. The method of claim 35 wherein the host cells in least one cycle are selected from S. cerevisiae cells and P. pastoris cells.

38. The method of claim 22, wherein the host cells in least one cycle are transformed by vectors comprising a mutant polynucleotide and a signal sequence that directs the secretion of polypeptides encoded by the mutant polynucleotide.

39. The method of claim 38, wherein the signal sequence in least one cycle is the PelB signal sequence.

40. The method of claim 22, wherein the screening step in least one cycle comprises screening for at least one of the following properties:
(a) the biological activity of the polypeptide;
(b) the stability of the polypeptide; or
(c) the yield of expressed polypeptide
(d) the yield of expressed functional polypeptide.

41. The method of claim 22, wherein screening in least one cycle comprises pre-screening for mutant colonies using nitrocellulose membranes.

42. The method of claim 22, wherein at least one of steps (ii) to (iv) in a repeated cycle differs from the corresponding step of a preceding cycle.

43. The method of claim 42, wherein at least one step for altering the parent nucleotide sequence in a repeated cycle differs from the corresponding step of a preceding cycle.

44. The method of claim 42, wherein the host cells used in a repeated cycle are different from the host cells in a preceding cycle.

45. The method of claim 44 wherein the host cells in one cycle are bacterial cells and in another cycle are yeast cells.

46. The method of claim 42, wherein the polypeptide property screened for in a repeated cycle is different from the polypeptide property screened for in a preceeding cycle.

47. The method of claim 42, wherein error-prone polymerase chain reaction is used for altering parent polynucleotide sequences in at least two cycles.

48. The method of claim 47, wherein the error rate in a repeated cycle is higher than the error rate in a preceding cycle.

49. The method of claim 48, wherein the error rate in a repeated cycle is about 4-6 mutations per polynucleotide, and the error rate in a preceding cycle is about 1-2 mutations per polynucleotide.

50. The method of claim 47, wherein the concentration of manganese ions employed in a repeated cycle is about 100 μM, and the concentration of manganese ions employed in a preceeding cycle is about 0.35 mM.

51. A functional polypeptide produced according to the method of claim 22.

52. A polynucleotide encoding for a horseradish peroxidase which has one or more mutations at an amino acid position selected from 371, 131, and 223, and wherein the starting methionine residue is at position 0.

53. A polynucleotide encoding for a horseradish peroxidase which has at least one mutation selected from L371I and L131P.

54. A polynucleotide encoding for a horseradish peroxidase which has mutations at amino acids 131 and 223, and at least one additional mutation at at an amino acid position selected from 47, 93, 102, 226, 241, 303, wherein the starting methionine residue is at position 0.

55. A polynucleotide encoding for a horseradish peroxidase which has the amino acid mutations L131P land L223Q, and at leats one additional mutation selected from N47S, R93L, T102A, P226Q, K241T, and V303E.

56. A mutant horseradish peroxidase having at least one mutation selected from L131P and L223Q, N47S, R93L, T102A, P226Q, K241T, and V303E.


SEQUENCE LISTING (30 sequences)

SEQ ID NO: 1 - DNA, 66 bp, Erwinia Carotovora
atgaaatacctattgcctacggcagccgctggattgttattactcgctgcccaaccagccatggcc

SEQ ID NO: 2 - protein, 22 aa, Erwinia Carotovora
MKYLLPTAAAGLLLLAAQPAMA

SEQ ID NO: 3 - DNA, 927 bp, Escherichia coli
atgcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactct

SEQ ID NO: 4 - protein, 309 aa, Escherichia coli
MQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNS

SEQ ID NO: 5 - DNA, 927 bp, Escherichia coli
atgcagttaacccctacattctacgacaatagctgtcccaacgtgtccaacatcgttcgcgacacaatcgtcaacgagctcagatccgatcccaggatcgctgcttcaataatacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactct

SEQ ID NO: 6 - protein, 309 aa, Escherichia coli
MQLTPTFYDNSCPNVSNIVRDTIVNELRSDPRIAASIIRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNS

SEQ ID NO: 7 - DNA, 927 bp, Escherichia coli
atgcagttaacccctacattctacgacaatagctgtcccaacgtgtccaacatcgttcgcgacacaatcgtcaacgagctcagatccgatcccaggatcgctgcttcaataatacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacatgtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactct

SEQ ID NO: 8 - protein, 309 aa, Escherichia coli
MQLTPTFYDNSCPNVSNIVRDTIVNELRSDPRIAASIIRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNMYYVNLEEQKGLIQSDQELFSSPNATDTIPLVRSFANSTQTFFNAFVEAMDRMGNITPLTGTQGQIRLNCRVVNSNS

SEQ ID NO: 9 - DNA, 927 bp, Escherichia coli
atgcagttaacccctacattctacgacaatagctgtcccaacgtgtccaacatcgttcgcgacacaatcgtcaacgagctcagatccgatcccaggatcgctgcttcaataatacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggacttagatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactct

SEQ ID NO: 10 - protein, 309 aa, Escherichia coli
MQLTPTFYDNSCPNVSNIVRDTIVNELRSDPRIAASIIRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDLDLRTPTIFDNKYYVNLEEQKGLIQSDQELFSSPNATDTIPLVRSFANSTQTFFNAFVEAMDRMGNITPLTGTQGQIRLNCRVVNSNS

SEQ ID NO: 11 - DNA, 927 bp, Escherichia coli
atgcagttaacccctacattctacgacaatagctgtcccaacgtgtccaacatcgttcgcgacacaatcgtcaacgagctcagatccgatcccaggatcgctgcttcaataatacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcccagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactct

SEQ ID NO: 12 - protein, 309 aa, Eschericia coli
MQLTPTFYDNSCPNVSNIVRDTIVNELRSDPRIAASIIRLHFHDCFVNGCDASILLDNTTSFRTEKDAFGNANSARGFPVIDRMKAAVESACPRTVSCADLLTIAAQQSVTLAGGPSWRVPLGRRDSLQAFPDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNS

SEQ ID NO: 13 - DNA, 927 bp, Escherichia coli
atgcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcccagatctggccaatgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatcagcggaccccaaccatcttcgataacaagtactatgtgaatctagaggagcagaaaggcctgatacagagtgatcaagaactgtttagcagtccaaacgccacagacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactct

SEQ ID NO: 14 - protein, 309 aa, Escherichia coli
MQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFPDLANANLPAPFFTLPQLKDSFRNVGLNRSSDLVALSGGHTFGKNQCRFIMDRLYNFSNTGLPDPTLNTTYLQTLRGLCPLNGNLSALVDFDQRTPTIFDNKYYVNLEEQKGLIQSDQELFSSPNATDTIPLVRSFANSTQTFFNAFVEAMDRMGNITPLTGTQGQIRLNCRVVNSNS

SEQ ID NO: 15 - DNA, 24 bp, Artificial Sequence
  Primer sequence
ttattgctcagcggtggcagcagc

SEQ ID NO: 16 - DNA, 24 bp, Artificial Sequence
  Primer sequence
aagcgctcatgagcccgaagtggc

SEQ ID NO: 17 - DNA, 927 bp, Escherichia coli
atgcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcccagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactct

SEQ ID NO: 18 - protein, 309 aa, Escherichia coli
MQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFPDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNS

SEQ ID NO: 19 - DNA, 927 bp, Escherichia coli
atgcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctggctatagctgcgcaacagagcgtgactcttgcaggcggaccgtcctggagagtgccgctcggtcgacgtgactccctacaggcattcccagatctggccaatgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatcagcggacccaaaccatcttcgataacaagtactatgtgaatctagaggagcagaaaggcctgatacagagtgatcaagaactgtttagcagtccaaacgccacagacaccatcccactggtgagaagttttgctaactctactcaaaccttctttaacgccttcgtggaagccatggaccgtatgggtaacattaccccactgacgggtacccaaggccagattcgtctgaactgcagagtggtcaacagcaactct

SEQ ID NO: 20 - protein, 309 aa, Escherichia coli
MQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLAIAAQQSVTLAGGPSWRVPLGRRDSLQAFPDLANANLPAPFFTLPQLKDSFRNVGLNRSSDLVALSGGHTFGKNQCRFIMDRLYNFSNTGLPDPTLNTTYLQTLRGLCPLNGNLSALVDFDQRTQTIFDNKYYVNLEEQKGLIQSDQELFSSPNATDTIPLVRSFANSTQTFFNAFVEAMDRMGNITPLTGTQGQIRLNCRVVNSNS

SEQ ID NO: 21 - DNA, 927 bp, Escherichia coli
atgcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccactaacagtcagttgtgcagacctgctggctatagctgcgcaacagagcgtgactcttgcaggcggaccgtcctggagagtgccgctcggtcgacgtgactccctacaggcattcccagatctggccaatgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatcagcggaccccaaccatcttcgataacaagtactatgtgaatctagaggagcagaaaggcctgatacagagtgatcaagaactgtttagcagtccaaacgccacagacaccatcccactggtgagaagttttgctaactctactcaaaccttctttaacgccttcgtggaagccatggaccgtatgggtaacattacccctctgacgggtacccaaggccagattcgtctgaactgcagagaggtcaacagcaactct

SEQ ID NO: 22 - protein, 309 aa, Escherichia coli
MQLTPTFYDNSCPNVSNIVRDTIVNELRSDPRIAASILRLHFHDCFVNGCDASILLDNTTSFRTEKDAFGNANSARGFPVIDRMKAAVESACPLTVSCADLLAIAAQQSVTLAGGPSWRVPLGRRDSLQAFPDLANANLPAPFFTLPQLKDSFRNVGLNRSSDLVALSGGHTFGKNQCRFIMDRLYNFSNTGLPDPTLNTTYLQTLRGLCPLNGNLSALVDFDQRTPTIFDNKYYVNLEEQKGLIQSDQELFSSPNATDTIPLVRSFANSTQTFFNAFVEAMDRMGNITPLTGTQGQIRLNCREVNSNS

SEQ ID NO: 23 - DNA, 927 bp, Escherichia coli
atgcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgagtggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctggctatagctgcgcaacagagcgtgactcttgcaggcggaccgtcctggagagtgccgctcggccgacgtgactccctacaggcattcccagatctggccaatgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatcagcggacccaaaccatcttcgataacaagtactatgtgaatctagaggagcagaaaggcctgatacagagtgatcaagaactgtttagcagtccaaacgccacagacaccatcccactggtgagaagttttgctaactctactcaaaccttctttaacgccttcgtggaagccatggaccgtatgggtaacattaccccactgacgggtacccaaggccagattcgtctgaactgcagagtggtcaacagcaactct

SEQ ID NO: 24 - protein, 309 aa, Escherichia coli; identical to SEQ ID NO: 20

SEQ ID NO: 25 - DNA, 18 bp, Artificial Sequence
  Primer sequence
cagttaacccctacattc

SEQ ID NO: 26 - DNA, 20 bp, Artificial Sequence
  Primer sequence
tgatgctgtcgccgaagaag

SEQ ID NO: 27 - DNA, 19 bp, Artificial Sequence
  Primer sequence
tcagttaacccctacattc

SEQ ID NO: 28 - DNA, 21 bp, Artificial Sequence
  Primer sequence
ccaccaccagtagagacatgg

SEQ ID NO: 29 - DNA, 22 bp, Artificial Sequence
  Primer sequence
gagaaaagagaggctgaagctc

SEQ ID NO: 30 - DNA, 20 bp, Artificial Sequence
  Primer sequence
tccttaccttccaataattc